(19) United States
(12) Patent Application Publication
(10) Pub. No.: US 2009/0214455 A1
(43) Pub. Date: Aug. 27, 2009

(54) PROCESS FOR MAKING UP OR CARING FOR KERATIN MATERIALS, COMPRISING THE APPLICATION OF COMPOUNDS A AND B, AT LEAST ONE OF WHICH IS SILICONE-BASED

(76) Inventors: Xavier Blin, Paris (FR); Jean Mondet, Aulnay Sous Bois (FR); Bruno Bavouzet, Gentilly (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/097,978

(22) PCT Filed: Dec. 20, 2006
(86) PCT No.: PCT/EP2006/069973

§ 371 Date: Feb. 13, 2009

Related U.S. Application Data
(60) Provisional application No. 60/754,283, filed on Dec. 29, 2005.
(60) Provisional application No. 60/818,271, filed on Jul. 5, 2006.

(30) Foreign Application Priority Data
Dec. 20, 2005 (FR) 0553977
Jun. 29, 2006 (FR) 0652707

(51) Int. Cl. A61K 8/22; A61Q 1/06; A61Q 1/10; A61Q 1/02

(52) U.S. Cl. 424/63; 424/64

(57) ABSTRACT

The invention relates to a cosmetic process for coating keratin materials, which consists in applying to the said keratin materials at least one compound A and at least one compound B, at least one of the compounds A and B being a silicone compound, the said compounds A and B being capable of reacting together via a hydrosilylation reaction, a condensation reaction, or crosslinking reaction in the presence of a peroxide.

The present invention relates to a process for coating keratin materials, in particular a non-therapeutic process for making up or caring for keratin materials, which consists in applying to the said keratin materials at least two compounds A and B, which are capable of reacting together, at least one of the compounds being silicone-based.

The compositions according to the invention may be compositions for making up or caring for keratin materials, in particular the skin, the lips, the eyelashes, the eyebrows or the nails.

Each composition may be a loose or compacted powder, a foundation, a makeup rouge, an eyeshadow, a concealer product, a blusher, a lipstick, a lip balm, a lip gloss, a lip pencil, an eye pencil, a mascara, an eyeliner, a body makeup product or a skin colouring product.

The care composition may be a care product for the eyelashes or the lips, or a care product for bodily and facial skin, especially an antisun product.

Lipstick and foundation compositions are commonly used to give the lips or the skin, and especially the face, an aesthetic colour. These makeup products generally contain fatty phases such as waxes and oils, pigments and/or fillers and optionally additives, for instance cosmetic or dermatological active agents.

When they are applied to the skin, these compositions have the drawback of transferring, i.e. of becoming at least partially deposited, and leaving marks, on certain supports with which they may come into contact and especially a glass, a cup, a cigarette, an item of clothing or the skin. This results in mediocre persistence of the applied film, making it necessary to regularly renew the application of the foundation or lipstick composition. Moreover, the appearance of these unacceptable marks, especially on blouse collars, may put certain women off using this type of makeup.

“Transfer-resistant” lip and skin makeup compositions are thus sought, which have the advantage of forming a deposit that does not become at least partially deposited onto the supports with which they come into contact (glass, clothing, cigarette or fabric) and show good staying power.

To limit the transfer of cosmetic compositions, it is known practice to use volatile oils. These volatile oils present in large amount render the makeup product, especially lipstick, uncomfortable for the user: the makeup deposit gives a sensation of drying-out and of tautness.

In the case of eyelash coating compositions or mascaras, anhydrous mascaras or mascaras with a low content of water and/or water-soluble solvents, known as “waterproof mascaras” are known in particular, which are formulated in the form of a dispersion of waxes in non-aqueous solvents and which show good resistance to water and/or to sebum.

However, the makeup film obtained after applying these compositions is not sufficiently resistant to water, for example when bathing or taking a shower, to tears or sweat, or to sebum. The mascara then has a tendency to wear away over time: grains are worn off and unattractive marks appear around the eyes.

The aim of the present invention is to provide a new route for formulating cosmetic compositions, especially makeup compositions, which makes it possible to obtain a film, deposited on keratin materials, which shows good transfer resistance and staying power properties over time, in particular resistance to water and rubbing, and a comfortable deposit on the skin, the lips, the eyelashes or the nails.

The inventors have discovered that it is possible to obtain such properties by using a system comprising silicone compounds that polymerize in situ so as to adhere better to keratin materials. These silicone compounds also show good biocompatibility.

The compounds A and B may be applied on keratin materials via several compositions comprising the compound(s) A and/or the compound(s) B alone or in mixture, or via one composition comprising said compound(s) A and/or compound(s) B

Accordingly, according to a first aspect, one subject of the present invention is a cosmetic process for coating keratin materials, which consists in applying to the said keratin materials at least one coat of a mixture of a first composition and of a second composition; the first and/or second composition comprising at least one compound A and/or at least one compound B and optionally at least a catalyst or a peroxide, at least one of the compounds A and B being a silicone compound, the said compounds A and B being capable of reacting together via a hydrosilylation reaction or a condensation reaction, or a crosslinking reaction in the presence of a peroxide, when they are placed in contact with each other, provided that the compounds A and B, and the catalyst when present or the peroxide, are not present together in the same compositions, the said mixture being obtained either extemporaneously before application to the keratin materials, or simultaneously with its application to the keratin materials.

These compounds are capable of reacting together on the keratin materials or on the support so as to form, on the keratin materials, an adherent film with good staying power.

According to one advantageous embodiment, compounds A and B are mixed together extemporaneously and the mixture is then applied to the keratin materials.

Accordingly, one subject of the invention is a cosmetic process for coating keratin materials, which consists in:
a. extemporaneously mixing together:
   at least one first composition and
   at least one second composition, the first and/or second composition comprising at least one compound A and/or at least one compound B and optionally at least a catalyst or a peroxide, at least one of the compounds A and B being a silicone compound, and the said compounds A and B being capable of reacting together via a hydrosilylation reaction or a condensation reaction, or a crosslinking reaction in the presence of a peroxide, when they are placed in contact with each other, provided that the compounds A and B, and the catalyst when present, or the peroxide, are not present together in the same compositions, and then
b. applying to the said keratin materials at least one coat of the said mixture.

According to one variant, compound A and compound B are applied via at least two different compositions, each comprising at least one compound A and/or at least one compound B and optionally at least a catalyst or a peroxide.

Accordingly, a subject of the present invention is a cosmetic process for coating keratin materials, the process comprising the application to the said keratin materials:
a. of at least one coat of a first composition;
b. of at least one coat of a second composition; the first and/or second composition comprising at least one compound A and/or at least one compound B and optionally at least a catalyst or a peroxide, at least one of the compounds A and B being a silicone compound, provided that the compounds A and B, and the catalyst when present, or the peroxide, are not present together in the same compositions, the said compounds A and B being capable of reacting together via a hydrosilylation reaction or a condensation reaction, or a crosslinking reaction in the presence of a peroxide, when they are placed in contact with each other.

In particular, each first and second composition contains at least one compound A and/or B

The terms first and second compositions do not in any way determine the order of application of said compositions on the keratinous materials.

Several coats of each of the first and second compositions may also be applied alternately to the keratin materials.

According to another aspect, a subject of the invention is a cosmetic composition for coating keratin materials, comprising
at least one compound A and at least one compound B, at least one of the compounds A and B being a silicone compound, the said compounds A and B being capable of reacting together via a hydrosilylation reaction or a condensation reaction, or a crosslinking reaction in the presence of a peroxide, when they are placed in contact with each other, and
at least one pigment other than carbon black and iron oxides.

According to one embodiment, at least one additional coat of at least one third composition comprising a cosmetically acceptable medium, and preferably at least one film-forming polymer and at least one organic (or oily) or aqueous solvent medium is applied onto the coats(s) of the composition(s) comprising compounds A and B in order, for example, to improve the staying power, gloss and/or comfort thereof.

According to another aspect, another subject of the present invention is a cosmetic kit comprising at least one compound A and/or at least one compound B and optionally at least a catalyst or a peroxide, at least one of the compounds A and B being a silicone compound, provided that the compounds A and B, and the catalyst when present, or the peroxide, are not present together in the same compositions, the said compounds A and B being capable of reacting together via a hydrosilylation reaction or a condensation reaction, or a crosslinking reaction in the presence of a peroxide, when they are placed in contact with each other.

According to one embodiment, at least a catalyst, as defined below, is applied on the keratinic materials to activate the reaction between the compound(s) A and/or the compound(s) B.

For example, the catalyst may be present in one the first or second composition applied on the keratinic materials, or in an additional composition, and the order of application of the composition does not matter.

According to one embodiment, at least one additional reactive compound, as defined here below, may be present in any one of the first and second composition, in both compositions or in an additional composition and the order of application of the compositions does not matter.

According to a preferred embodiment, the first composition comprises at least one compound A and at least one compound B, and the second composition comprises at least one compound A and a catalyst.

According to one embodiment, the kit also comprises a composition for removing the coating obtained on the keratin materials by reaction of compounds A and B, the said composition preferably comprising at least one organic solvent or oil chosen from the organic solvents and oils described later in point II.

Each composition may be packaged separately in the same packaging article, for example in a two-compartment pen, the base composition being delivered from one end of the pen and the top composition being delivered from the other end of the pen, each end being closed, especially in a leaktightness manner, with a cap. Each composition may also be packaged in the same packaging article, the mixing of the two compositions being performed at the end(s) of the packaging article during the delivery of each composition.

Alternatively, each of the first and second compositions may be packaged in a different packaging article.

A subject of the invention is also the use of a kit as described above for obtaining a film deposited on keratin materials, which shows improved staying power, gloss and/or comfort properties.

Needless to say, each composition comprises a cosmetically acceptable medium, i.e. a non-toxic medium that may be applied to human keratin materials and that has a pleasant appearance, odour and feel.

### I/ Compounds A and B

The term “silicone compound” means a compound comprising at least two organosiloxane units. According to a specific embodiment, the compound A and the compound B are silicon based.

The compounds A and B may be amine based or not.

According to one embodiment, at least one of the compounds A and B is a polymer whose main chain is mainly formed of organosiloxane units.

Among the silicones mentioned above, some may have both film-forming and adhesive properties, depending, for example, on their proportion of silicone or depending on whether they are used as a mixture with a particular additive. It is consequently possible to modify the film-forming properties or the adhesive properties of such compounds according to the intended use: this is in particular the case for the “room-temperature vulcanization” reactive elastomeric silicones.

The compounds A and B may react together at a temperature ranging from room temperature (20° C.±5° C.) to 180° C. Preferably, A and B are able to react at room temperature and atmospheric pressure, preferably in the presence of a catalyst, by a hydrosilylation reaction or a condensation reaction, or a crosslinking reaction in the presence of a peroxide

According to an embodiment, the compound A and/or the compound B may contain a polar group able to form at least one hydrogen bond with the keratin materials. The term <<polar group>> is understood to mean a group comprising carbon and hydrogen atoms and at least a heteroatom (such as O, N, S or P), such that said group is able to form at least one hydrogen bond with the keratin materials.

Compounds carrying at least a polar group able to form at least one hydrogen bond are particularly advantageous as they contribute, to the composition comprising them, a better adhesion to the keratin materials, thanks to the ability of these groups to form a hydrogen bond with the keratin materials.

The polar group(s) carried by at least one of the compounds A and B is/are able to form a hydrogen bond and comprise either a hydrogen atom bonded to an electronegative atom, or an electronegative atom such as oxygen, nitrogen or sulphur atom. When the group comprises a hydrogen atom bonded to an electronegative atom, the hydrogen atom can interact with the another electronegative atom carried, for example, by another molecule, such as the keratin, to form a hydrogen bond. When the group comprises an electronegative atom, the electronegative atom can interact with the hydrogen atom bonded to an electronegative atom carried, for example, by another molecule, such as the keratin, to form a hydrogen bond.

Advantageously, these polar groups may be chosen from the following groups:
carboxylic acid —COOH,
amino —NR₁R₂, with R₁ et R₂ identical or different represent an alkyl radical comprising from 1 to 6 carbon atoms or one of the R₁ et R₂ radicals represent a hydrogen group,
pyridino,
amido of formulae —NH—COR′ or —CO—NH—R′ in which R′ represents a hydrogen atom or an alkyl radical comprising from 1 to 6 carbon atoms
pyrrolidino preferably chosen from groups of formulae:

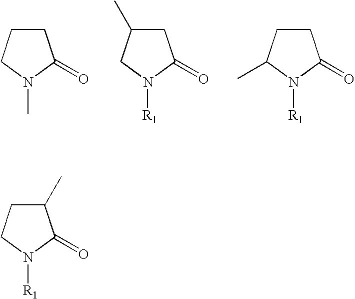

Where R₁ is an alkyl radical comprising from 1 to 6 carbon atoms,
carbamoyl of formulae —O—CO—NH—R′ or —NH—CO—OR′, R′ being as defined above,
thiocarbamoyl, such as —O—CS—NH—R′ or —NH—CS—O R′, R′ being as defined above,
ureyl such as —NR′ —CO—N(R′)₂, the R′being identical or different and being as defined above,
sulfonamido such as —NR′—S(═O)₂—R′, R′being as defined above,
   and their associations.

Preferably, these polar groups are present in an amount lower than or equal 10% by weight relative to the total weight of each of the compound A or B, preferably lower than or equal 5% by weight, for example in an amount ranging from 1 to 3% by weight relative to the weight of each compound A or B.

The polar group(s) can be carried in the main chain of the compound A and/or B or can be present at the chain end or on the side position with respect to the said chain.

### 1/ Compounds A and B Capable of Reacting Via Hydrosilylation

According to one embodiment, the silicone compounds are capable of reacting via hydrosilylation, this reaction being able to be represented schematically, in a simplified manner, as follows:

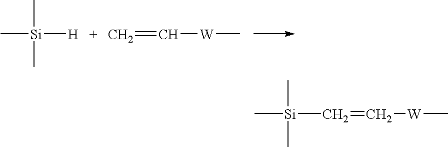

where W represents a carbon or silicon based chain containing one or several unsaturated aliphatic groups.

In this case, compound A may be chosen from silicone compounds comprising at least two unsaturated aliphatic groups. For example compound A may comprise a silicon main chain whose unsaturated aliphatic groups are pendent to the main chain (side group) or located at the ends of the main chain of the compound (end group).

These particular compounds are called polyorganosiloxanes with unsaturated aliphatic groups in the following specification.

According to an embodiment, the compound A and/or the compound B carry a polar group, as described above, that is able to form a hydrogen bond with keratin materials. This polar group is preferably carried by the compound A comprising at least unsaturated aliphatic groups.

According to one embodiment, compound A is chosen from polyorganosiloxanes comprising at least two unsaturated aliphatic groups, for example two or three vinyl or allylic groups, each linked to a silicon atom.

According to one advantageous embodiment, compound A is chosen from polyorganosiloxanes comprising siloxane units of formula:

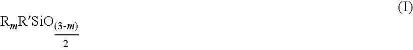

in which:
R represents a linear or cyclic monovalent hydrocarbon-based group containing from 1 to 30 carbon atoms, preferably from 1 to 20 carbon atoms, and better from 1 to 10 carbon atoms, for instance a short-chain alkyl radical containing, for example, from 1 to 10 carbon atoms, in particular a methyl radical or a phenyl group, preferably a methyl group,
m is equal to 1 or 2, and
R′represents:
   an unsaturated aliphatic hydrocarbon group containing from 2 to 10 carbon atoms, preferably from 2 to 5 carbon atoms, for instance a vinyl group or a group R″—CH═CHR′″ in which R″ is a divalent aliphatic hydrocarbon chain linked to the silicon atom, containing from 1 to 8 carbon atoms, and R′″ is a hydrogen atom or an alkyl radical containing from 1 to 4 carbon atoms, preferably a hydrogen atom; groups R′that may be mentioned include vinyl and allyl groups and mixtures thereof or
   an unsaturated cyclic hydrocarbon-based group containing from 5 to 8 carbon atoms, for instance a cyclohexenyl group.

Preferably, R′is an unsaturated aliphatic hydrocarbon group, preferably a vinyl group.

According to a specific embodiment, the polyorganosiloxane comprise also units of formula

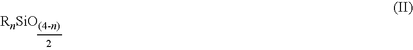

in which R is a group as defined above and n is equal to 1, 2 or 3.

According to one variant, the compound A may comprise a silicon resin comprising at least two ethylenic unsaturations, the said resin being capable of reacting with compound B via hydrosilylation. Examples that may be mentioned include resins of MQ or MT type, themselves bearing —CH═CH₂ unsaturated reactive end groups.

These resins are crosslinked organosiloxane polymers.
The nomenclature of silicone resins is known under the name “MDTQ”, the resin being described as a function of the various siloxane monomer units it comprises, each of the letters “MDTQ” characterizing a type of unit.
The letter M represents the monofunctional unit of formula (CH₃)₃SiO_{1/2}, the silicon atom being linked to only one oxygen atom in the polymer comprising this unit.
   The letter D means a difunctional unit (CH₃)₂SiO_{2/2} in which the silicon atom is linked to two oxygen atoms.
   The letter T represents a trifunctional unit of formula (CH₃)SiO_{3/2}.
   In the units M, D and T defined above, at least one of the methyl groups may be substituted with a group R other than a methyl group, such as a hydrocarbon-based radical (especially alkyl) containing from 2 to 10 carbon atoms or a phenyl group, or alternatively a hydroxyl group.

Finally, the letter Q means a tetrafunctional unit SiO_{4/2} in which the silicon atom is linked to four hydrogen atoms, which are themselves linked to the rest of the polymer,

As examples of such resins may be cited the MT silicon resins such as poly(phenyl-vinylsilsesquioxane) for instance commercialised by Gelest under the reference SST-3PV1.

Preferably, the compounds A comprise from 0.01% to 1% by weight of unsaturated aliphatic groups.

Advantageously, the compound A is chosen from polyorganosiloxanes, specifically polyorganosiloxanes containing siloxane units of formulae (I) and (II) as described above.

Compound B preferably comprises at least two free Si—H groups (hydrogenosilane groups).

Compound B may be chosen advantageously from organosiloxanes comprising at least one alkylhydrogenosiloxane units of the following formula:

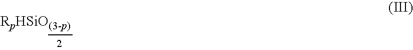

in which:
R represents a linear or cyclic monovalent hydrocarbon-based group containing from 1 to 30 carbon atoms, for instance an alkyl radical containing from 1 to 30 carbon atoms, preferably from 1 to 20 carbon atoms and better from 1 to 10 carbon atoms, in particular a methyl radical, or a phenyl group, and p is equal to 1 or 2.
R is preferably a hydrocarbon-based group, preferably a methyl radical.

The organosiloxanes compounds B containing alkylhydrogenosiloxane units may also comprise units of formula:

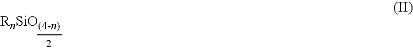

as defined above.

The compound B can be a silicon resin comprising at least one unit chosen from M, D and T units as defined above and comprising at least one Si—H group, such as as poly(methyl-hydridosilsesquioxane) for instance commercialised by Gelest under the reference SST-3 MH1.1.

Preferably, the organosiloxanes compounds B comprise from 0.5% to 2.5% by weight of groups Si—H.

Advantageously, the radicals R represent a methyl group in the above formulae (I), (II) and (III).

Preferably, the organosiloxanes B comprise end groups of formula (CH₃)₃SiO_{1/2}.

Advantageously, the organosiloxanes B comprise at least two alkylhydrogenosiloxane units of formula (H₃C)HSiO and optionally comprise units (H₃C)₂SiO.

Such organosiloxanes compounds B with hydrogenosiloxane units are described for example in document EP0465744.

According to one variant, compound A is chosen from organic oligomers or polymers (the term “organic” means compounds whose main chain is not silicone-based) or from hybrid organic/silicone polymers or oligomers, the said polymers or oligomers bearing at least two reactive unsaturated aliphatic groups, compound B being chosen from the hydrogenosiloxanes mentioned above.

According to one embodiment, the compounds A of organic nature or hybrid organic/silicone nature comprising at least two unsaturated aliphatic reactive groups, comprise at least a polar group as described above.

Compound A, of organic nature, may then be chosen from vinyl or (meth)acrylic polymers or oligomers, polyesters, polyurethanes and/or polyureas, polyethers, perfluoropolyethers, polyolefins such as polybutene or polyisobutylene, dendrimers and organic hyperbranched polymers, or mixtures thereof.

In particular, the organic polymer or the organic part of the hybrid polymer may be chosen from the following polymers:
a) ethylenically unsaturated polyesters:

This is a group of polymers of polyester type containing at least two ethylenic double bonds, randomly distributed in the main polymer chain. These unsaturated polyesters are obtained by polycondensation of a mixture
of linear or branched aliphatic or cycloaliphatic dicarboxylic acids especially containing from 3 to 50 carbon atoms, preferably from 3 to 20 carbon atoms and better, from 3 to 10 carbon atoms, such as adipic acid or sebacic acid, of aromatic dicarboxylic acids especially containing from 8 to 50 carbon atoms, preferably from 8 to 20 carbon atoms and better, from 8 to 14 carbon atoms, such as phthalic acids, especially terephthalic acid, and/or of dicarboxylic acids derived from ethylenically unsaturated fatty acid dimers such as the oleic or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 carbon atoms, and better from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 carbon atoms, and better, from 6 to 15 carbon atoms, such as bisphenol A and bisphenol B, and/or of diol dimers obtained from the reduction of fatty acid dimers as defined above, and
of one or more dicarboxylic acids or anhydrides thereof comprising at least one polymerizable ethylenic double bond and containing from 3 to 50 carbon atoms, preferably from 3 to 20 carbon atoms, and better from 3 to 10 carbon atoms, such as maleic acid, fumaric acid or itaconic acid.
b) polyesters containing (meth)acrylate side groups and/or end groups:

This is a group of polymers of polyester type obtained by polycondensation of a mixture
of linear or branched aliphatic or cycloaliphatic dicarboxylic acids especially containing from 3 to 50 carbon atoms, preferably from 3 to 20 carbon atoms, and better, from 3 to 10 carbon atoms, such as adipic acid or sebacic acid, of aromatic dicarboxylic acids especially containing from 8 to 50 carbon atoms, preferably from 8 to 20 carbon atoms, and better, from 8 to 14 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Unichema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 carbon atoms, and better, from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 carbon atoms, and better, from 6 to 15 carbon atoms, such as bisphenol A and bisphenol B, and
of at least one ester of (meth)acrylic acid and of a diol or polyol containing from 2 to 20 carbon atoms and preferably from 2 to 6 carbon atoms, such as 2-hydroxyethyl(meth)acrylate, 2-hydroxypropyl(meth)acrylate or glycerol methacrylate.

These polyesters differ from those described above in point a) by the fact that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50,000: molar mass 1500, on average 6 acrylate functions per molecule)
c) polyurethanes and/or polyureas containing (meth)-acrylate groups, obtained by polycondensation
   of aliphatic, cycloaliphatic and/or aromatic diisocyanates, triisocyanates and/or polyiso-cyanates especially containing from 4 to 50 and preferably from 4 to 30 carbon atoms, such as hexamethylene diisocyanate, isophorone diiso-cyanate, toluene diisocyanate, diphenylmethane diisocyanate or isocyanurates of formula

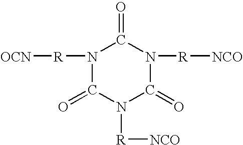

resulting from the trimerization of 3 molecules of diisocyanates OCN—R—CNO, in which R is a linear, branched or cyclic hydrocarbon-based radical comprising from 2 to 30 carbon atoms;
of polyols, especially of diols, free of polymerizable ethylenic unsaturations, such as 1,4-butanediol, ethylene glycol or trimethylol-propane, and/or of aliphatic, cycloaliphatic and/or aromatic polyamines, especially diamines, especially containing from 3 to 50 carbon atoms, such as ethylenediamine or hexamethylenediamine, and
of at least one ester of (meth)acrylic acid and of a diol or polyol containing from 2 to 20 carbon atoms and preferably from 2 to 6 carbon atoms, such as 2-hydroxyethyl(meth)acrylate, 2-hydroxypropyl(meth)acrylate or glycerol methacrylate.

Such polyurethane/polyureas containing acrylate groups are sold, for example, under the name SR 368 (tris(2-hydroxyethyl) isocyanurate-triacrylate) or Craynor® 435 by the company Cray Valley, or under the name Ebecryl® by the company UCB (Ebecryl® 210: molecular mass 1500, 2 acrylate functions per molecule, Ebecryl® 230: molecular mass 5000, 2 acrylate functions per molecule, Ebecryl® 270: molecular mass 1500, 2 acrylate functions per molecule, Ebecryl® 8402: molecular mass 1000, 2 acrylate functions per molecule, Ebecryl® 8804: molecular mass 1300, 2 acrylate functions per molecule, Ebecryl® 220: molecular mass 1000, 6 acrylate functions per molecule, Ebecryl® 2220: molecular mass 1200, 6 acrylate functions per molecule, Ebecryl® 1290: molecular mass 1000, 6 acrylate functions per molecule, Ebecryl® 800: molecular mass 800, 6 acrylate functions per molecule).

Mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB.
d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10,000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley.
e) epoxyacrylates obtained by reaction between
   at least one diepoxide chosen, for example, from:
      bisphenol A diglycidyl ether,
      a diepoxy resin resulting from the reaction between bisphenol A diglycidyl ether and epichlorohydrin,
      an epoxy ester resin containing α,ω-diepoxy end groups resulting from the condensation of a dicarboxylic acid containing from 3 to 50 carbon atoms with a stoichiometric excess of (i) and/or (ii), and
      an epoxy ether resin containing α,ω-diepoxy end groups resulting from the condensation of a diol containing from 3 to 50 carbon atoms with a stoichiometric excess of (i) and/or (ii),
      natural or synthetic oils bearing at least 2 epoxide groups, such as epoxidized soybean oil, epoxidized linseed oil or epoxidized vernonia oil,
      a phenol-formaldehyde polycondensate (Novolac® resin), the end groups and/or side groups of which have been epoxidized,
         and
   one or more carboxylic acids or polycarboxylic acids comprising at least one ethylenic double bond in the α,β-position relative to the carboxylic group, for instance (meth)acrylic acid or crotonic acid or esters of (meth)acrylic acid and of a diol or polyol containing from 2 to 20 carbon atoms and preferably from 2 to 6 carbon atoms, such as 2-hydroxyethyl(meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel.
f) poly(C₁₋₅₀ alkyl(meth)acrylates) comprising at least two functions containing an ethylenic double bond borne by the hydrocarbon-based side chains and/or end chains.
   Such copolymers are sold, for example, under the names IRR® 375, OTA® 480 and Ebecryl® 2047 by the company UCB.
g) polyolefins such as polybutene or polyisobutylene,
h) perfluoropolyethers containing acrylate groups obtained by esterification, for example with (meth)acrylic acid, of perfluoropolyethers bearing hydroxyl side groups and/or end groups.

Such α,ω-diol perfluoropolyethers are described especially in EP-A-1 057 849 and are sold by the company Ausimont under the name Fomblin® Z Diol.
i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

Dendrimers (from the Greek dendron=tree) are “arborescent”, i.e. highly branched, polymer molecules invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech.

Hyperbranched polymers are polycondensates, generally of polyester, polyamide or polyethyleneamine type, obtained from multifunctional monomers, which have an arborescent structure similar to that of dendrimers but are much less regular than dendrimers (see, for example, WO-A-93/17060 and WO 96/12754).

The company Perstorp sells hyperbranched polyesters under the name Boltorn®. Hyperbranched polyethylene-amines will be found under the name Comburst® from the company Dendritech. Hyperbranched poly(esteramides) containing hydroxyl end groups are sold by the company DSM under the name Hybrane®.

These hyperbranched dendrimers and polymers esterified or amidated with acrylic acid and/or methacrylic acid are distinguished from the polymers described in points a) to h) above by the very large number of ethylenic double bonds present. This high functionality, usually greater than 5, makes them particularly useful by allowing them to act as “crosslinking nodes”, i.e. sites of multiple crosslinking.

These dendritic and hyperbranched polymers may thus be used in combination with one or more of the polymers and/or oligomers a) to h) above.

### 1.a Additional Reactive Compound

According to one embodiment, at least one of the compositions comprising the compounds A and/or B may also comprise an additional reactive compound comprising at least two unsaturated aliphatic groups, for instance:
organic or mineral particles comprising at the surface at least two unsaturated aliphatic groups: examples that may be mentioned include silicas that have been surface-treated, for example with silicone compounds containing vinyl groups, for instance cyclotetramethyltetravinylsiloxane-treated silica,
silazane compounds such as hexamethyldisilazane.

### 1.b Catalyst

The hydrosilylation reaction is advantageously performed in the presence of a catalyst that may be present in one of the compositions comprising compound A and/or compound B or in a separate composition, the catalyst preferably being platinum-based or tin-based.

Examples that may be mentioned include platinum-based catalysts deposited on a support of silica gel or charcoal (coal) powder, platinum chloride, platinum salts and chloroplatinic acids.

Chloroplatinic acids in hexahydrate or anhydrous form, which are readily dispersible in organosilicone media, are preferably used.

Mention may also be made of platinum complexes such as those based on chloroplatinic acid hexahydrate and on divinyltetramethyldisiloxane.

The catalyst may be present in the composition(s) in a content ranging from 0.0001% to 20% by weight relative to the total weight of the composition comprising it.

Polymerization inhibitors, and more particularly catalyst inhibitors, may also be introduced into the compositions of the invention, in order to increase the stability of the composition over time or to retard the polymerization. Non-limiting examples that may be mentioned include cyclic polymethylvinylsiloxanes, in particular tetravinyl tetramethyl cyclotetrasiloxane,acetylenic alcohols, preferably volatile, such as methylisobutynol.

The presence of ionic salts in one and/or the other of the compositions may have an influence on the rate of polymerization of the compounds.

Examples of a combination of such compounds A and B reacting via hydrosilylation that may be mentioned include the following references sold by the company Dow Corning: DC7-9800 Soft Skin Adhesive parts A & B, and the mixture prepared by Dow Corning

Part X:

| | | amounts | |
|---|---|---|---|
| Ingredient (INCI name) | CAS N^{o} | (%) | Role |
| Dimethyl Siloxane, | 68083-19-2 | 55-95 | Polymer |
| Dimethylvinylsiloxy- | | | |
| terminated | | | |
| Silica Silylate | 68909-20-6 | 10-40 | Filler |
| 1,3-Diethenyl-1,1,3,3- | 68478-92-2 | Trace | Catalyst |
| Tetramethyldisiloxane | | | |
| complexes | | | |
| Tetramethyldivinyldisiloxane | 2627-95-4 | 0.1-1 | Polymer |

Part Y

| | | amounts | |
|---|---|---|---|
| Ingredient (INCI name) | CAS N^{o} | (%) | role |
| Dimethyl Siloxane, | 68083-19-2 | 55-95 | Polymer |
| Dimethylvinylsiloxy- | | | |
| terminated | | | |
| Silica Silylate | 68909-20-6 | 10-40 | Filler |
| Dimethyl, Methylhydrogen | 68037-59-2 | 1-10 | Polymer |
| Siloxane, | | | |
| trimethylsiloxy- | | | |
| terminated | | | |

Advantageously, compounds A and B are chosen from silicone compounds capable of reacting via hydrosilylation; in particular, compound A is chosen from polyorganosiloxanes comprising units of formula (I) described above and compound B is chosen from organosiloxanes comprising alkylhydrogenosiloxane units of formula (III) described above.

According to a specific embodiment, compound A is a polydimethylsiloxane with vinyl end groups, and compound B is a methylhydrogenosiloxane.

According to a particular embodiment, compound A contains at least one polar group.

### 2/Compounds A and B Capable of Reacting Via Condensation

According to another embodiment, the silicone compounds A and B are capable of reacting via condensation, either in the presence of water (hydrolysis) by reaction of two compounds bearing alkoxysilane groups, or via “direct” condensation by reaction of a compound bearing (an) alkoxysilane group(s) and a compound bearing (a) silanol group(s) or by reaction of two compounds bearing (a) silanol group(s).

When the condensation is performed in the presence of water, this water may in particular be the ambient humidity, residual water on the skin, the lips, the eyelashes and/or the nails, or water provided by an external source, for example by premoistening the keratin materials (for example with a mister or natural or artificial tears).

In this case of condensation reaction, compounds A and B, which may be identical or different, may thus be chosen from silicone compounds comprising at least two alkoxysilane groups and/or at least two silanol groups (Si—OH), laterally and/or at the end of a chain.

According to one embodiment, the compound A and/or the compound B carry at least a polar group able to form a hydrogen bond with keratin materials, as described above.

According to one advantageous embodiment, compounds A and/or B are chosen from polyorganosiloxanes comprising at least two alkoxysilane groups.

The term “alkoxysilane” means a group comprising at least a —Si—OR part, R being an alkyl radical comprising from 1 to 6 carbon atoms.

The compounds A and B are in particular chosen from polyorganosiloxanes comprising alkoxysilane end groups, more specifically those comprising at least two alkoxysilane end groups, preferably trialkoxysilane end groups.

These compounds A and/or B preferably predominantly comprise units of formula

R⁹ₛSiO_{(4-s)/2} (IV)

in which R⁹ independently represents a radical chosen from alkyl groups containing from 1 to 6 carbon atoms, phenyl and fluoroalkyl groups, and s is equal to 0, 1, 2 or 3. Preferably, R⁹ represents independently an alkyl group containing from 1 to 6 carbon atoms.

Alkyl groups that may especially be mentioned include methyl, ethyl, propyl, butyl and hexyl, and mixtures thereof, preferably methyl or ethyl.

A fluoroalkyl group that may be mentioned is 3,3,3-trifluoropropyl.

According to a specific embodiment, compounds A and B, which may be identical or different, are polyorganosiloxanes comprising units of formula

(R⁹₂SiO₂)_{f}— (V)

in which R⁹ is as described above, preferably R⁹ is a methyl radical, and f is such that the polymer has advantageously a viscosity at 25° C. ranging from 0.5 to 3000 Pa·s and preferably ranging from 5 to 150 Pa·s, for example f can range from 2 to 5000, preferably from 3 to 3000 and better from 5 to 1000.

The polyorganosiloxane compounds A and B advantageously comprise at least two trialkoxysilane end groups per polymer molecule, the said groups having the following formula

—ZSiR¹ₓ(OR)₃₋ₓ, (VI)

in which:
the radicals R independently represent a methyl, ethyl, n-propyl, isopropyl, n-butyl, sec-butyl or isobutyl group,
R¹ is a methyl or ethyl group,
x is equal to 0 or 1, and preferably x is equal to 0, and
Z is chosen from: divalent hydrocarbon-based groups free of ethylenic unsaturation and containing from 2 to 18 carbon atoms (alkylene groups), combinations of divalent hydrocarbon-based groups and of siloxane segments of formula (IX):

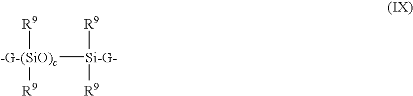

R⁹ being as described above, G is a divalent hydrocarbon-based radical free of ethylenic unsaturation and containing from 2 to 18 carbon atoms and c is an integer ranging from 1 to 6.
Z and G may be chosen especially from alkylene groups such as ethylene, propylene, butylene, pentylene and hexylene, and arylene groups such as phenylene.

Preferably, Z is an alkylene group, and better still ethylene.

These polymers can present an average number of 1,2 trialkoxysilane end groups or end chains per molecule, preferably at least 1,5 trialkoxysilane end groups per molecule. These polymers can present an average number of 1,2 trialkoxysilane end groups or end chains per molecule, some polymers may comprise other types of end groups such as end groups of formula CH═CH—SiR⁹₂— or R⁶₂—Si—, in which R⁹ is as defined above and each R⁶ group is independently chosen from R⁹ or vinyle groups. Examples of such end groups are trimethoxysilane, triethoxysilane, vinyledimethoxysilane and vinylemethoxysiphenylsilane.

Such polymers are especially described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the content of which is incorporated by reference into the present patent application.

Mention may be made as compounds A and B, in particular of the polymer of formula

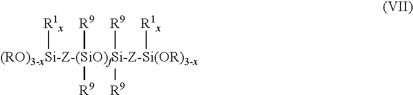

in which R, R¹, R⁹, Z, x and f are as described above.

Compounds A and/or B may also comprise a mixture of polymer of formula (VII) above with polymers of formula (VIII) below:

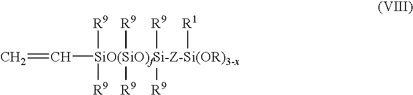

in which R, R¹, R⁹, Z, x and f are as described above.

When the polyorganosiloxane containing alkoxysilane group(s) comprises such a mixture, the various polyorganosiloxanes are present in contents such that the organosilyl end chains represent less than 40% and preferably less than 25% by number of the end chains.

The A and/or B polyorganosiloxanes compounds that are particularly preferred are those of formula (VII) described above.

Such compounds A and/or B are described for example in document WO 01/96450.

As indicated hereinabove, compounds A and B may be identical or different.

According to one embodiment, compounds A and B represent a mixture of polydimethylsiloxane with methoxysilane groups.

According to one variant, one of the two reactive compounds A and/or B is of silicone nature and the other is of organic nature. For example, compound A is chosen from organic oligomers or polymers and hybrid organic/silicone oligomers or polymers, the said polymers or oligomers comprising at least two alkoxysilane groups, and B is chosen from silicone compounds such as the polyorganosiloxanes described above. In particular, the organic oligomers or polymers are chosen from vinyl or (meth)acrylic oligomers or polymers, polyesters, polyamides, polyurethanes and/or polyureas, polyethers, polyolefins, perfluoro-polyethers, dendrimers and hyperbranched organic polymers, and mixtures thereof.

According to a particular embodiment, compound A of organic nature or hybrid organic/silicone nature contains at least one polar group as described above.

The organic polymers of vinyl or (meth)acrylic nature, bearing alkoxysilane side groups, may be obtained in particular by copolymerization with a (meth)acryloxy-propyltrimethoxysilane and, a vinyltrimethoxysilane, a vinyltriethoxysilane, an allyltrimethoxysilane, etc. Such (meth)acrylic polymers are described in the document of KUSABE.M, Pitture e Verniei—European Coating; 12-B, pages 43-49, 2005, and in particular polyacrylates with alkoxysilane groups with the reference MAX of Kaneka or described in the publication of PROBSTER, M, Adhesion-Kleben & Dichten, 2004, 481 (1-2), pages 12-14.

The organic polymers A resulting from a polycondensation or a polyaddition, such as polyesters, polyamides, polyurethanes and/or polyureas or poly-ethers, and bearing alkoxysilane side and/or end groups, may result, for example, from the reaction with one of the following silane co-reagents bearing an alkoxysilane group: aminopropyltrimethoxysilane, amino-propyltriethoxysilane, aminoethylaminopropyltri-methoxysilane, glycidoxypropyltrimethoxysilane, glycidoxypropyltriethoxysilane, epoxycyclohexylethyl-trimethoxysilane, mercaptopropyltrimethoxysilane.

Examples of polyethers and polyisobutylene bearing alkoxysilane groups are described in the document of KUSABE.M, Pitture e Verniei—European Coating; 12-B, pages 43-49, 2005.

Examples of polyurethane bearing alkoxysilane groups are described in the document of PROBSTER, M, Adhesion-Kleben & Dichten, 2004, 481 (1-2), pages 12-14 or in the document of LANDON, S., Pitture e Verniei vol. 73, N^{o} 11, pages 18-24, 1997 or in the document of HUANG, Mowo, Pitture e Verniei vol. 5, 2000, pages 61-67, mention may be made of polyurethane bearing alkoxysilane groups from OSI-WITCO-GE.

As examples of A and/or B polyorganosiloxanes compounds may be cited resins of MQ or MT type, themselves bearing alkoxysilane and/or silanol end groups, for instance poly(isobutylsilsesquioxane) resins with silanol functional groups, such as the products sold by Gelest under the reference SST-S7C41 (3 Si—OH groups)

### 2.a Additional Reactive Compound

At least one of the compositions may also comprise an additional reactive compound comprising at least two alkoxysilane or silanol groups. Examples that may be mentioned include one or more organic or mineral particles comprising at their surface alkoxysilane and/or silanol groups, for example fillers surface-treated with such groups.

### 2.b Catalyst

The condensation reaction may be performed in the presence of a metal-based catalyst, which may be present in the one of the compositions comprising A and/or B, or in a separate composition

The catalyst useful in this reaction is preferably a titanium-based catalyst. Mention may be made especially of tetraalkoxytitanium-based catalysts of formula

Ti(OR²)_{y}(OR³)_{4-y},

in which R² is chosen from tertiary alkyl radicals such as tert-butyl, tert-amyl and 2,4-dimethyl-3-pentyl; R³ represents an alkyl radical containing from 1 to 6 carbon atoms, preferably a methyl, ethyl, n-propyl, isopropyl, n-butyl, sec-butyl or hexyl group and y is a number ranging from 3 to 4 and better still from 3.4 to 4.

The catalyst may be present in on or the other composition(s), in particular in the first or the second composition, in a content ranging from 0.0001% to 20% by weight relative to the total weight of the composition comprising it.

### 2.c Diluent

The compositions used in the invention, in particular the first and/or the second composition, may also comprise a volatile silicone oil (or diluent) intended to reduce the viscosity of the composition. This oil may be chosen from linear short-chain silicones such as hexamethyldisiloxane, octamethyltrisiloxane, cyclic silicones such as octamethylcyclotetrasiloxane or decamethylcyclopentasiloxane, and mixtures thereof.

This silicone oil may represent from 5% to 95% and preferably from 10% to 80% by weight relative to the weight of each composition.

As examples of a combination of compounds A and B bearing alcoxysilane groups and reacting via condensation reaction, mention may be made of the combination of the following mixtures X′ and Y′ prepared by Dow Corning

Part X′:

| | | Amount | |
|---|---|---|---|
| Ingredient (INCI name) | CAS N^{o} | (%) | Role |
| Bis-Trimethoxysiloxyethyl | PMN87176 | 25-45 | Polymer |
| Tetramethyldisiloxyethyl | | | |
| Dimethicone (1) | | | |
| Silica Silylate | 68909- | 5-20 | Filler |
| | 20-6 | | |
| Disiloxane | 107-46-0 | 30-70 | Solvent |

Part Y′:

| | | Amount | |
|---|---|---|---|
| Ingredient (INCI name) | CAS N^{o} | (%) | Role |
| Disiloxane | 107-46-0 | 80-99 | Solvent |
| Tetra T Butyl Titanate | — | 1-20 | Catalyst |

It is to be noted that the compounds A and B are identical in the mixture XI.

### 3/ Crosslinking in the Presence of Peroxide:

This reaction is preferably performed by heating to a temperature of greater than or equal to 50° C. and preferably greater than or equal to 80° C., going up to 120° C.

Compounds A and B, which may be identical or different, comprise in this case at least two side groups —CH₃ and/or at least two side chains bearing a group —CH₃.

Compounds A and B are preferably silicone-based and may be chosen, for example, from non-volatile linear polydimethylsiloxanes with a degree of polymerization of greater than 6, containing at least two —CH₃ side groups and/or at least two side chains bearing a group —CH₃.

Examples of such polymers are described in the document “Reactive Silicone” from Gelest Inc., Edition 2004, page 6, et in particular vinylmethylsiloxane-dimethylsiloxane copolymers (also called gums) with a molecular weight ranging from 500 000 to 900 000 and in particular with a viscosity greater than 2 000 000 cSt.

As peroxides that may be used in the context of the invention, mention may be made of benzoyl peroxide and 2,4-dichlorobenzoyl peroxide, and mixtures thereof.

According to one embodiment, the hydrosilylation reaction, the condensation reaction or the crosslinking reaction in the presence of a peroxide between compounds A and B is accelerated by supplying heat, for example by raising the temperature of the system to between 25° C. and 180° C. The system will especially react on the skin.

In general, irrespective of the type of reaction via which compounds A and B react together, the mole percentage of compound A relative to the compounds A and B, i.e. the ratio A/(A+B)×100 may range from 5% to 95%, preferably from 10% to 90% and better still from 20% to 80%.

Similarly, the mole percentage of compound B relative to the compounds B and A, i.e. the ratio B/(A+B)×100, may range from 5% to 95%, preferably from 10% to 90% and better still from 20% to 80%.

Compound A may have a weight-average molecular mass (Mw) ranging from 150 to 1 000 000, preferably from 200 to 800 000 and more preferably from 200 to 250 000.

Compound B may have a weight-average molecular mass (Mw) ranging from 200 to 1 000 000, preferably 300 to 800 000 and more preferably from 500 to 250 000.

Compound A may represent from 0.15% to 95%, preferably from 1% to 90% and better still from 5% to 80% by weight relative to the total weight of the composition comprising it, in particular relative to the weight of each of the first composition or second composition, or relative to the total weight of the composition when A and B are present in the same composition.

Compound B may represent from 0.15% to 95%, preferably from 1% to 90% and better still from 5% to 80% by weight relative to the total weight of the composition comprising it, in particular relative to the weight of the each of the first composition or second composition, or relative to the total weight of the composition when A and B are present in the same composition.

The ratio between compounds A and B may be varied so as to modify the rate of reaction and thus the rate of formation of the film or so as to adapt the properties of the formed film (for example its adhesive properties) according to the desired use.

In particular, compounds A and B may be present in a ratio A/B ranging from 0.05 to 20 and better still from 0.1 to 10.

According to one embodiment, at least one of the compositions may comprise silica, especially synthetic silica surface-treated with a hydrophobic agent (preferably a silicon agent), in particular fumed silica subjected to a hydrophobic surface treatment, such as the silicas described below as fillers and/or gelling agents.

### II/ Liquid Fatty Phase

At least one of the first and second compositions advantageously comprises a liquid fatty phase.

For the purposes of the present patent application, the term “liquid fatty phase” means a fatty phase that is liquid at room temperature (25° C.) and atmospheric pressure (760 mmHg), composed of one or more mutually compatible non-aqueous fatty substances that are liquid at room temperature, also known as organic solvents or oils.

The oil may be chosen from volatile oils and/or non-volatile oils, and mixtures thereof.

The oil may be present in a content ranging from 1% to 90% by weight and preferably from 5% to 50% by weight relative to the total weight of each first and second composition.

For the purposes of the invention, the term “volatile oil” means an oil that is capable of evaporating on contact with the skin or the keratin materials in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

The term “non-volatile oil” means an oil that remains on the skin or the keratin materials at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C8-C16 alkanes, for instance C8-C16 isoalkanes of petroleum origin (also known as isoparaffins), for instance isododecane (also known as 2,2,4,4,6-pentamethylheptane) isodecane and isohexadecane, for example the oils sold under the trade names Isopar or Permethyl, branched C8-C16 esters and isohexyl neopentanoate, and mixtures thereof. Other volatile hydrocarbon-based oils, for instance petroleum distillates, especially those sold under the name Shell Solt by the company Shell, may also be used. The volatile solvent is preferably chosen from volatile hydrocarbon-based oils containing from 8 to 16 carbon atoms, and mixtures thereof.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethyl-cyclopentasiloxane, dodecamethylcyclohexasiloxane, heptamethylhexyltrisiloxane, heptamethyloctyltri-siloxane, hexamethyldisiloxane, octamethyltrisiloxane, decamethyltetrasiloxane and dodecamethylpentasiloxane, and mixtures thereof.

Mention may also be made of the linear volatile alkyltrisiloxane oils of general formula (I):

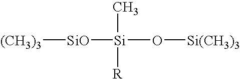

in which R represents an alkyl group containing from 2 to 4 carbon atoms and of which one or more hydrogen atoms may be substituted with one or more fluorine or chlorine atoms.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluorinated solvents such as nonafluoro-methoxybutane or perfluoromethylcyclopentane may also be used.

The oil of formula (I) for which R is an ethyl group is especially sold under the name Baysilone TP 3886 and the oil for which R is a butyl group is especially sold under the name Baysilone TP 3887 by the company Bayer Silicones.

Preferably, the compositions used in the process according to the invention each have a volatile oil content of less than or equal to 50% by weight, preferably less than or equal to 30% and better still less than or equal to 10% by weight relative to the total weight of each first and second composition. More preferably, the first and second composition(s) are free of volatile oil.

According to one advantageous embodiment, at least one of the first and second compositions used in the process according to the invention comprises at least one non-volatile oil, chosen in particular from non-volatile hydrocarbon-based oils and/or silicone oils and/or fluoro oils.

Non-volatile hydrocarbon-based oils that may especially be mentioned include:
hydrocarbon-based oils of plant origin, such as triesters of fatty acids and of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or caprylic/capric acid triglycerides, for instance those sold by the company Stearineries Dubois or those sold under the names Miglyol 810, 812 and 818 by the company Dynamit Nobel;
synthetic ethers containing from 10 to 40 carbon atoms;
apolar hydrocarbon-based oils, for instance squalene, linear or branched hydrocarbons such as liquid paraffin, liquid petroleum jelly and naphthalene oil, hydrogenated or partially hydrogenated polyisobutene, isoeicosane, squalane, decene/butene copolymers and polybutene/polyisobutene copolymers, especially Indopol L-14, and polydecenes such as Puresyn 10, and mixtures thereof;
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance Purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate or diisostearyl malate; and pentaerythritol esters;
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpenta-decanol;
higher fatty acids such as oleic acid, linoleic acid or linolenic acid;
carbonates;
acetates;
citrates;
and mixtures thereof.

The non-volatile silicone oils may be:
non-volatile polydimethylsiloxanes (PDMS),
polydimethylsiloxanes comprising alkyl or alkoxy groups, which are pendent and/or at the end of a silicone chain, these groups each containing from 3 to 40 carbon atoms,
phenylsilicones, for instance phenyl trimethi-cones, phenyl dimethicones, phenyltrimethylsiloxy-diphenylsiloxanes, diphenyl dimethicones, diphenyl-methyldiphenyltrisiloxanes and 2-phenylethyl trimethyl-siloxysilicates;
optionally fluorinated polyalkylmethylsiloxanes, for instance polymethyltrifluoropropyldimethyl-siloxanes,
polyalkylmethylsiloxanes substituted with functional groups such as hydroxyl, thiol and/or amine groups;
polysiloxanes modified with fatty acids, fatty alcohols or polyoxyalkylenes,
and mixtures thereof.

According to one embodiment, the liquid fatty phase comprises an ester oil. This ester oil may be chosen from the esters of monocarboxylic acids with monoalcohols and polyalcohols.

Advantageously, the said ester corresponds to formula (IV) below:

R₁—CO—O—R₂ (IV)
where R₁ represents a linear or branched alkyl radical of 1 to 40 carbon atoms and preferably of 7 to 19 carbon atoms, optionally comprising one or more ethylenic double bonds, and optionally substituted,
R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted.

The term “optionally substituted” means that R₁ and/or R₂ can bear one or more substituents chosen, for example, from groups comprising one or more hetero atoms chosen from O, N and S, such as amino, amine, alkoxy and hydroxyl.

Preferably, the total number of carbon atoms of R₁+R₂ is ≧9.

R₁ may represent the residue of a linear or, preferably, branched fatty acid, preferably a higher fatty acid, containing from 1 to 40 and even better from 7 to 19 carbon atoms, and R₂ may represent a linear or, preferably, branched hydrocarbon-based chain containing from 1 to 40, preferably from 3 to 30 and even better from 3 to 20 carbon atoms. Once again, preferably the number of carbon atoms of R₁+R₂≧9.

Examples of groups R₁ are those derived from fatty acids chosen from the group consisting of acetic acid, propionic acid, butyric acid, caproic acid, caprylic acid, pelargonic acid, capric acid, undecanoic acid, lauric acid, myristic acid, palmitic acid, stearic acid, isostearic acid, arachidic acid, behenic acid, oleic acid, linolenic acid, linoleic acid, oleostearic acid, arachidonic acid and erucic acid, and mixtures thereof.

Examples of esters include purcellin oil (cetostearyl octanoate), isononyl isononanoate, isopropyl myristate, 2-ethylhexyl palmitate, 2-octyldodecyl stearate, 2-octyldodecyl erucate, isostearyl isostearate, and heptanoates, octanoates, decanoates or ricinoleates of alcohols or polyalcohols, for example of fatty alcohols.

Advantageously, the esters are chosen from the compounds of formula (IV) above, in which R₁ represents an unsubstituted linear or branched alkyl group of 1 to 40 carbon atoms and preferably of 7 to 19 carbon atoms, optionally comprising one or more ethylenic double bonds, and R₂ represents an unsubstituted linear or branched alkyl group of 1 to 40 carbon atoms, preferably of 3 to 30 carbon atoms and even better of 3 to 20 carbon atoms, optionally comprising one or more ethylenic double bonds.

Preferably, R₁ is an unsubstituted branched alkyl group of 4 to 14 carbon atoms and preferably of 8 to 10 carbon atoms, and R₂ is an unsubstituted branched alkyl group of 5 to 15 carbon atoms and preferably of 9 to 11 carbon atoms. Preferably, in formula (I), R₁—CO— and R₂ have the same number of carbon atoms and are derived from the same radical, preferably an unsubstituted branched alkyl, for example isononyl, i.e. the ester oil molecule is advantageously symmetrical.

The ester oil will preferably be chosen from the following compounds:
isononyl isononanoate,
cetostearyl octanoate,
isopropyl myristate,
2-ethylhexyl palmitate,
2-octyldodecyl stearate,
2-octyldodecyl erucate,
isostearyl isostearate.

Advantageously, the non-volatile oil is chosen from the ester oils of formula (IV) above and phenyl silicones, and mixtures thereof.

Accordingly, according to one embodiment, a subject of the invention is a cosmetic composition for coating keratin materials, comprising
i) at least one compound A and at least one compound B, at least one of the compounds A and B being a silicone compound, the said compounds A and B being capable of reacting together via a hydrosilylation or condensation reaction, or a crosslinking reaction in the presence of a peroxide, when they are placed in contact with each other, and
ii) at least one non-volatile oil chosen from:
   the esters corresponding to formula (IV) below:

R₁—CO—O—R₂ (IV)
where R₁ represents a linear or branched alkyl radical of 1 to 40 carbon atoms and preferably of 7 to 19 carbon atoms, optionally comprising one or more ethylenic double bonds, and optionally substituted,
R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted,
   phenyl silicones, and
mixtures thereof.

The non-volatile oil may be present in a content ranging from 0.1% to 80% by weight, preferably from 1% to 60% by weight, better still from 5% to 50% by weight and even better still from 14% to 40% by weight relative to the total weight of each first and second composition or relative to the total weight of the composition when A and B are present in the same composition.

When the first and second compositions are intended to be applied to the lips, a “viscous” oil may be used in particular, i.e. an oil whose viscosity at 25° C. is advantageously greater than or equal to 200 cSt, especially greater than or equal to 500 cSt or even greater than or equal to 1000 cSt. The viscous oil advantageously has a molecular mass of greater than or equal to 600 g/mol, for example greater than or equal to 700, or even 800, or even 900 g/mol.

The dynamic viscosity at 25° C. of the viscous oil may be measured with a Mettler RM 180 rotary viscometer, taking into account the density of the oil in order to make the conversion into cSt.

The Mettler RM 180 machine (Rheomat) may be equipped with different spindles depending on the order of magnitude of the viscosity that it is desired to measure. For a viscosity of between 0.18 and 4.02 Pa·s, the machine is equipped with a No. 3 spindle. For a viscosity of between 1 and 24 Pa·s, the machine is equipped with a No. 4 spindle, and for a viscosity of between 8 and 122 Pa·s, the machine is equipped with a No. 5 spindle. The viscosity is read on the machine in deviation units (DU). Reference is then made to charts provided with the measuring machine to obtain the corresponding value in poises, and then to convert it into stokes.

The spin speed of the spindle is 200 rpm.

Once the spindle is in rotation, at a constant set spin speed (in the present case 200 rpm), the viscosity value of the oil may vary over time. Measurements are taken at regular time intervals until they become constant. The viscosity value that has become constant over time is the value retained as being the dynamic viscosity value of the viscous oil.

This oil may be chosen from the silicone oils or apolar hydrocarbon-based oils with a viscosity of greater than or equal to 200 cSt mentioned above.

According to one embodiment, the first and second compositions used in the process according to the invention are anhydrous.

### Aqueous Phase

At least one of the first and second compositions may comprise an aqueous phase.

The aqueous phase may consist essentially of water; it may also comprise a mixture of water and/or of water-miscible solvent (miscibility in water of greater than 50% by weight at 25° C.), for instance lower monoalcohols containing from 1 to 5 carbon atoms, such as ethanol or isopropanol, glycols containing from 2 to 8 carbon atoms, such as propylene glycol, ethylene glycol, 1,3-butylene glycol or dipropylene glycol, C₃-C₄ ketones and C₂-C₄ aldehydes, and mixtures thereof.

The aqueous phase (water and optionally the water-miscible solvent) may be present in a content ranging from 5% to 95% by weight, preferably from 10% to 85% by weight and better still from 2% to 80% by weight relative to the total weight of each composition.

### Solid or Pasty Fatty Substances

At least one of the first and second compositions of the process according to the invention may also comprise at least one fatty substance that is solid at room temperature, chosen especially from waxes and pasty fatty substances, and mixtures thereof. These fatty substances may be of animal, plant, mineral or synthetic origin.

### Wax

At least one of the first and second compositions according to the invention may comprise a wax or a mixture of waxes.

The wax under consideration in the context of the present invention is in general a lipophilic compound, which is solid at room temperature (25° C.), with a reversible solid/liquid change of state, having a melting point of greater than or equal to 30° C. that may be up to 120° C.

By bringing the wax to the liquid state (melting), it is possible to make it miscible with the oils and to form a microscopically homogeneous mixture, but on returning the temperature of the mixture to room temperature, recrystallization of the wax in the oils of the mixture is obtained.

In particular, the waxes that are suitable for the invention may have a melting point of greater than about 45° C. and in particular greater than 55° C.

The melting point of the wax may be measured using a differential scanning calorimeter (DSC), for example the calorimeter sold under the name DSC 30 by the company Mettler.

The measuring protocol is as follows:

A 15 mg sample of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature rise ranging from 0° C. to 120° C. at a heating rate of 5° C./minute. During the second temperature rise, the variation of the difference in power absorbed by the empty crucible and by the crucible containing the sample of product is measured as a function of the temperature. The melting point of the compound is the temperature value corresponding to the top of the peak of the curve representing the variation in the difference in power absorbed as a function of the temperature.

The waxes that may be used in the first and second compositions according to the invention are chosen from waxes that are solid and rigid at room temperature, of animal, plant, mineral or synthetic origin, and mixtures thereof.

The wax may also have a hardness ranging from 0.05 MPa to 30 MPa and preferably ranging from 6 MPa to 15 MPa. The hardness is determined by measuring the compression force, measured at 20° C. using the texturometer sold under the name TA-TX2i by the company Rheo, equipped with a stainless-steel cylinder 2 mm in diameter travelling at a measuring speed of 0.1 mm/s, and penetrating the wax to a penetration depth of 0.3 mm.

The measuring protocol is as follows:

The wax is melted at a temperature equal to the melting point of the wax +20° C. The molten wax is poured into a container 30 mm in diameter and 20 mm deep. The wax is recrystallized at room temperature (25° C.) for 24 hours and is then kept at 20° C. for at least 1 hour before performing the hardness measurement. The hardness value is the maximum compression force measured divided by the surface area of the texturometer cylinder in contact with the wax.

Hydrocarbon-based waxes, for instance beeswax, lanolin wax or Chinese insect wax; rice wax, carnauba wax, candelilla wax, ouricury wax, esparto grass wax, cork fibre wax, sugarcane wax, Japan wax and sumach wax; montan wax, microcrystalline waxes, paraffins and ozokerite; polyethylene waxes, the waxes obtained by Fisher-Tropsch synthesis and waxy copolymers, and also esters thereof, may especially be used.

Mention may also be made of waxes obtained by catalytic hydrogenation of animal or plant oils containing linear or branched C₈-C₃₂ fatty chains.

Among these waxes that may especially be mentioned are hydrogenated jojoba oil, isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S by the company Heterene, bis(1,1,1-trimethylolpropane) tetrabehenate sold under the name Hest 2T-4B by the company Heterene.

Mention may also be made of silicone waxes, for instance alkyl or alkoxy dimethicones containing from 16 to 45 carbon atoms, and fluoro waxes.

The wax obtained by hydrogenation of olive oil esterified with stearyl alcohol, sold under the name Phytowax Olive 18 L57 or else the waxes obtained by hydrogenation of castor oil esterified with cetyl alcohol sold under the names Phytowax ricin 16L64 and 22L73 by the company Sophim may also be used. Such waxes are described in Patent Application FR-A-2 792 190.

According to one particular embodiment, the first and second compositions according to the invention may comprise at least one “tacky” wax, i.e. a wax with a tack of greater than or equal to 0.7 N·s and a hardness of less than or equal to 3.5 MPa.

The tacky wax used may especially have a tack ranging from 0.7 N·s to 30 N·s, in particular greater than or equal to 1 N·s, especially ranging from 1 N·s to 20N·s, in particular greater than or equal to 2 N·s, especially ranging from 2 N·s to 10 N·s and in particular ranging from 2 N·s to 5 N·s.

The tack of the wax is determined by measuring the change in force (compression force or stretching force) as a function of time, at 20° C., using the texturometer sold under the name TA-TX2i® by the company Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N·s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is measured according to the protocol described previously.

A tacky wax that may be used is a C₂₀-C₄₀ alkyl (hydroxystearyloxy)stearate (the alkyl group containing from 20 to 40 carbon atoms), alone or as a mixture, in particular a C₂₀-C₄₀ alkyl 12-(12′-hydroxystearyloxy)-stearate.

Such a wax is especially sold under the names Kester Wax K 82 P® and Kester Wax K 80 P® by the company Koster Keunen.

The waxes mentioned above generally have a starting melting point of less than 45° C.

The wax(es) may be in the form of an aqueous microdispersion of wax. The expression “aqueous microdispersion of wax” means an aqueous dispersion of wax particles in which the size of the said wax particles is less than or equal to about 1 μm.

Wax microdispersions are stable dispersions of colloidal wax particles, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of a portion of water, followed by gradual addition of hot water with stirring. The intermediate formation of an emulsion of the water-in-oil type is observed, followed by a phase inversion, with final production of a microemulsion of the oil-in-water type. On cooling, a stable microdispersion of solid wax colloidal particles is obtained.

The wax microdispersions may also be obtained by stirring the mixture of wax, surfactant and water using stirring means such as ultrasound, high-pressure homogenizers or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles consist essentially of a wax or a mixture of waxes. However, they may comprise a small proportion of oily and/or pasty fatty additives, a surfactant and/or a common liposoluble additive/active agent.

The term “pasty fatty substance” means a lipophilic fatty compound comprising at a temperature of 23° C. a liquid fraction and a solid fraction.

The said pasty compound preferably has a hardness at 20° C. ranging from 0.001 to 0.5 MPa and preferably from 0.002 to 0.4 MPa.

The hardness is measured according to a method of penetration of a probe in a sample of compound and in particular using a texture analyser (for example the TA-XT2i machine from Rheo) equipped with a stainless-steel spindle 2 mm in diameter. The hardness measurement is performed at 20° C. at the centre of five samples. The spindle is introduced into each sample at a pre-speed of 1 mm/s and then at a measuring speed of 0.1 mm/s, the penetration depth being 0.3 mm. The hardness value revealed is that of the maximum peak.

The liquid fraction of the pasty compound measured at 23° C. preferably represents 9% to 97% by weight of the compound. This liquid fraction at 23° C. preferably represents between 15% and 85% and more preferably between 40% and 85% by weight. The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in solid crystalline form. The pasty compound is said to be in the liquid state when all of its mass is in liquid form.

The heat of fusion of the pasty compound is equal to the area under the curve of the thermogram obtained using a differential scanning calorimeter (DSC), such as the calorimeter sold under the name MDSC 2920 by the company TA Instrument, with a temperature rise of 5 or 10° C. per minute, according to standard ISO 11357-3:1999. The heat of fusion of the pasty compound is the amount of energy required to make the compound change from the solid state to the liquid state. It is expressed in J/g.

The heat of fusion consumed at 23° C. is the amount of energy absorbed by the sample to change from the solid state to the state that it has at 23° C., consisting of a liquid fraction and a solid fraction.

The liquid fraction of the pasty compound, measured at 32° C., preferably represents from 30% to 100% by weight of the compound, preferably from 80% to 100% and more preferably from 90% to 100% by weight of the compound. When the liquid fraction of the pasty compound measured at 32° C. is equal to 100%, the temperature of the end of the melting range of the pasty compound is less than or equal to 32° C.

The liquid fraction of the pasty compound measured at 32° C. is equal to the ratio of the heat of fusion consumed at 32° C. to the heat of fusion of the pasty compound. The heat of fusion consumed at 32° C. is calculated in the same manner as the heat of fusion consumed at 23° C.

The pasty substances are generally hydrocarbon-based compounds, for instance lanolins and derivatives thereof, or alternatively PDMSs.

The nature and amount of the solid substances depend on the desired mechanical properties and textures. As a guide, the waxes may represent from 0.1% to 70% by weight, better still from 1% to 40% and even better still from 5% to 30% by weight relative to the total weight of each composition.

### Film-Forming Polymer

At least one of the first and second compositions may comprise a film-forming polymer. According to the present invention, the term “film-forming polymer” means a polymer capable, by itself or in the presence of an auxiliary film-forming agent, of forming a continuous film that adheres to a support and especially to keratin materials.

The film-forming polymer may be present in a solids content (or active material content) ranging from 0.1% to 30% by weight, preferably from 0.5% to 20% by weight and better still from 1% to 15% by weight relative to the total weight of each composition.

Among the film-forming polymers that may be used in the composition of the present invention, mention may be made of synthetic polymers, of free-radical type or of polycondensate type, and polymers of natural origin, and mixtures thereof.

The expression “free-radical film-forming polymer” means a polymer obtained by polymerization of unsaturated and especially ethylenically unsaturated monomers, each monomer being capable of homopoly-merizing (unlike polycondensates).

The film-forming polymers of free-radical type may be, in particular, vinyl polymers or copolymers, in particular acrylic polymers.

The vinyl film-forming polymers may result from the polymerization of ethylenically unsaturated monomers containing at least one acidic group and/or esters of these acidic monomers and/or amides of these acidic monomers.

Monomers bearing an acidic group which may be used are α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), especially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl(meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates that may be mentioned are hydroxyethyl acrylate, 2-hydroxypropyl acrylate, hydroxyethyl methacrylate and 2-hydroxypropyl methacrylate.

Among the aryl(meth)acrylates that may be mentioned are benzyl acrylate and phenyl acrylate.

The (meth)acrylic acid esters that are particularly preferred are the alkyl(meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned above.

Examples of vinyl esters that may be mentioned are vinyl acetate, vinyl neodecanoate, vinyl pivalate, vinyl benzoate and vinyl t-butylbenzoate.

Styrene monomers that may be mentioned are styrene and α-methylstyrene.

Among the film-forming polycondensates that may be mentioned are polyurethanes, polyesters, polyester-amides, polyamides, epoxyester resins and polyureas.

The polyurethanes may be chosen from anionic, cationic, nonionic and amphoteric polyurethanes, polyurethane-acrylics, polyurethane-polyvinylpyrrolidones, poly-ester-polyurethanes, polyether-polyurethanes, polyureas and polyurea/polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azeleic acid, suberic acid, sebacic acid, fumaric acid, maleic acid, itaconic acid, phthalic acid, dodecanedioic acid, 1,3-cyclo-hexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylol-propane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

The polyester may also comprise at least one monomer bearing at least one group —SO₃M, with M representing a hydrogen atom, an ammonium ion NH₄⁺ or a metal ion such as, for example, an Na⁺, Li⁺, K⁺, Mg²⁺, Ca²⁺, Cu²⁺, Fe²⁺ or Fe³⁺ ion. A difunctional aromatic monomer comprising such a group —SO₃M may be used in particular.

The aromatic nucleus of the difunctional aromatic monomer also bearing a group —SO₃M as described above may be chosen, for example, from benzene, naphthalene, anthracene, biphenyl, oxybiphenyl, sulfonylbiphenyl and methylenebiphenyl nuclei. As examples of difunctional aromatic monomers also bearing a group —SO₃M, mention may be made of: sulfoisophthalic acid, sulfotereph-thalic acid, sulfophthalic acid, 4-sulfonaphthalene-2,7-dicarboxylic acid.

The copolymers preferably used are those based on isophthalate/sulfoisophthalate, and more particularly copolymers obtained by condensation of diethylene glycol, cyclohexanedimethanol, isophthalic acid and sulfoisophthalic acid.

The polymers of natural origin, optionally modified, may be chosen from shellac resin, sandarac gum, dammar resins, elemi gums, copal resins and cellulose polymers, and mixtures thereof.

According to a first embodiment of the invention, the film-forming polymer may be a water-soluble polymer and may be present in an aqueous phase of the first and/or second composition; the polymer is thus solubilized in the aqueous phase of the composition.

According to another variant, the film-forming polymer may be a polymer dissolved in a liquid fatty phase comprising organic solvents or oils such as those described above (the film-forming polymer is thus said to be a liposoluble polymer). The liquid fatty phase preferably comprises a volatile oil, optionally mixed with a non-volatile oil, the oils possibly being chosen from those mentioned above.

Examples of liposoluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octane-dioate, divinyl dodecanedioate and divinyl octadecane-dioate.

Examples of these copolymers that may be mentioned are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctan-oate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinyl-benzene, vinyl acetate/1-octadecene, crosslinked with 0.2% divinylbenzene, and allyl propionate/allyl stearate, crosslinked with 0.2% divinylbenzene.

Examples of liposoluble film-forming polymers that may also be mentioned are liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from polyvinyl stearate, polyvinyl stearate crosslinked with the aid of divinylbenzene, of diallyl ether or of diallyl phthalate, polystearyl(meth)acrylate, poly-vinyl laurate and polylauryl(meth)acrylate, it being possible for these poly(meth)acrylates to be cross-linked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging from 2000 to 500 000 and preferably from 4000 to 200 000.

Mention may also be made of liposoluble homopolymers, and in particular those resulting from the homopolymerization of vinyl esters containing from 9 to 22 carbon atoms or of alkyl acrylates or methacrylates, the alkyl radicals containing from 2 to 24 carbon atoms.

Examples of liposoluble homopolymers that may especially be mentioned include: polyvinyl laurate and polylauryl(meth)acrylates, these poly(meth)acrylates possibly being crosslinked using ethylene glycol dimethacrylate or tetraethylene glycol dimethacrylate.

According to one advantageous embodiment, the first and/or second composition of the process according to the invention comprises at least one polyvinyl laurate film-forming polymer.

As liposoluble film-forming polymers which may be used in the invention, mention may also be made of polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the copolymers of VP/vinyl acetate, VP/ethyl methacrylate, butylated polyvinyl-pyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomen-clature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned include those sold:
by the company Wacker under the reference Resin MK, such as Belsil PMS MK;
by the company Shin-Etsu under the reference KR-220L.

Siloxysilicate resins that may be mentioned include trimethyl siloxysilicate (TMS) resins such as those sold under the reference SR 1000 by the company General Electric or under the reference TMS 803 by the company Wacker. Mention may also be made of the trimethyl siloxysilicate resins sold in a solvent such as cyclomethicone, sold under the name KF-7312J by the company Shin-Etsu, and DC 749 and DC 593 by the company Dow Corning.

Mention may also be made of silicone resin copolymers such as those mentioned above with polydimethyl-siloxanes, for instance the pressure-sensitive adhesive copolymers sold by the company Dow Corning under the reference Bio-PSA and described in document U.S. Pat. No. 5,162,410, or the silicone copolymers derived from the reaction of a silicone resin, such as those described above, and of a diorganosiloxane, as described in document WO 2004/073 626.

According to one embodiment of the invention, the film-forming polymer is a film-forming linear block ethylenic polymer, which preferably comprises at least a first block and at least a second block with different glass transition temperatures (Tg), the said first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in document EP 1 411 069 or WO 04/028 488.

The film-forming polymer may also be present in the composition in the form of particles dispersed in an aqueous phase or in a non-aqueous solvent phase, which is generally known as a latex or pseudolatex. The techniques for preparing these dispersions are well known to those skilled in the art.

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432 by the company Dow Chemical, Daitosol 5000 AD or Daitosol 5000 SJ® by the company Daito Kasey Kogyo; Syntran 5760® by the company Interpolymer, Allianz OPT by the company Rohm & Haas, aqueous dispersions of acrylic or styrene/acrylic polymers sold under the brand name Joncryl by the company Johnson Polymer, or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Sancure 861®, Sancure 878® and Sancure 2060® by the company Goodrich, Impranil 85® by the company Bayer and Aquamere H-1511® by the company Hydromer; the sulfopolyesters sold under the brand name Eastman AQ® by the company Eastman Chemical Products, and vinyl dispersions, for instance Mexomer PAM from the company Chimex, and mixtures thereof.

Examples of non-aqueous film-forming polymer dispersions that may also be mentioned include acrylic dispersions in isododecane, for instance Mexomer PAP® from the company Chimex, and dispersions of particles of a grafted ethylenic polymer, preferably an acrylic polymer, in a liquid fatty phase, the ethylenic polymer advantageously being dispersed in the absence of additional stabilizer at the surface of the particles as described especially in document WO 04/055 081.

The composition according to the invention may comprise a plasticizer that promotes the formation of a film with the film-forming polymer. Such a plasticizer may be chosen from any compound known to those skilled in the art as being capable of fulfilling the desired function.

### Dyestuffs

At least one of the first and second compositions used in the process according to the invention may comprise at least one dyestuff chosen, for example, from pigments, nacres, dyes and materials with an effect, and mixtures thereof.

These dyestuffs may be present in a content ranging from 0.01% to 50% by weight and preferably from 0.01% to 30% by weight relative to the weight of each first and second composition or relative to the total weight of the composition when A and B are present in the same composition.

The pigments that are useful in the present invention may be in the form of powder or of pigmentary paste.

The term “dyes” should be understood as meaning compounds, generally organic, which are soluble in at least one oil or in an aqueous-alcoholic phase.

The term “pigments” should be understood as meaning white or coloured, mineral or organic particles, which are insoluble in an aqueous medium, and which are intended to colour and/or opacify the resulting film.

The term “nacres” or nacreous pigments should be understood as meaning coloured particles of any form, which may or may not be iridescent, especially produced by certain molluscs in their shell or else synthesized, and which have a colour effect via optical interference.

The pigment may be an organic pigment. The term “organic pigment” means any pigment that satisfies the definition in Ullmann's encyclopaedia in the chapter on organic pigments. The organic pigment may especially be chosen from nitroso, nitro, azo, xanthene, quinoline, anthraquinone, phthalocyanin, metal complex, isoindolinone, isoindoline, quinacridone, perinone, perylene, diketopyrrolopyrrole, thioindigo, dioxazine, triphenylmethane and quinophthalone compounds.

The organic pigment(s) may be chosen, for example, from carmine, carbon black, aniline black, melanin, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenolic derivatives as described in patent FR 2 679 771.

These pigments may also be in the form of composite pigments as described in patent EP 1 184 426. These composite pigments may be composed especially of particles comprising an inorganic nucleus at least partially coated with an organic pigment and at least one binder to fix the organic pigments to the nucleus.

Examples that may also be mentioned include pigmentary pastes of organic pigments such as the products sold by the company Hoechst under the names:
Jaune Cosmenyl IOG: Pigment Yellow 3 (CI 11710);
Jaune Cosmenyl G: Pigment Yellow 1 (CI 11680);
Orange Cosmenyl GR: Pigment Orange 43 (CI 71105);
Rouge Cosmenyl R″: Pigment Red 4 (CI 12085);
Carmine Cosmenyl FB: Pigment Red 5 (CI 12490);
Violet Cosmenyl RL: Pigment Violet 23 (CI 51319);
Bleu Cosmenyl A2R: Pigment Blue 15.1 (CI 74160);
Vert Cosmenyl GG: Pigment Green 7 (CI 74260);
Noir Cosmenyl R: Pigment Black 7 (CI 77266).

The pigment may also be a lake. The term “lake” means insolubilized dyes adsorbed onto insoluble particles, the assembly thus obtained remaining insoluble during use.

The inorganic substrates onto which the dyes are adsorbed are, for example, alumina, silica, calcium sodium borosilicate or calcium aluminium borosilicate, and aluminium.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090).

An example of a lake that may be mentioned is the product known under the following name: D&C Red 7 (CI 15 850:1).

The pigment may also be a pigment with special effects. The term “pigments with special effects” means pigments that generally create a non-uniform coloured appearance (characterized by a certain shade, a certain vivacity and a certain lightness) that changes as a function of the conditions of observation (light, temperature, observation angles, etc.). They thus contrast with white or coloured pigments that afford a standard uniform opaque, semi-transparent or transparent shade.

Two types of pigment with special effects exist: those with a low refractive index, such as fluorescent, photochromic or thermochromic pigments, and those with a high refractive index, such as nacres or flakes.

Pigments with special effects that may be mentioned include nacreous pigments such as white nacreous pigments such as mica coated with titanium or with bismuth oxychloride, coloured nacreous pigments such as titanium mica with iron oxides, titanium mica especially with ferric blue or with chromium oxide, titanium mica with an organic pigment of the abovementioned type, and also nacreous pigments based on bismuth oxychloride.

Mention may also be made of pigments with an interference effect that are not fixed onto a substrate, for instance liquid crystals (Helicones HC from Wacker), holographic interference flakes (Geometric Pigments or Spectra f/x from Spectratek). Pigments with special effects also comprise fluorescent pigments, whether these are substances that are fluorescent in daylight or that produce an ultraviolet fluorescence, phosphorescent pigments, photochromic pigments, thermochromic pigments and quantum dots, sold, for example, by the company Quantum Dots Corporation.

Quantum dots are luminescent semiconductive nanoparticles capable of emitting, under light excitation, irradiation with a wavelength of between 400 nm and 700 nm. These nanoparticles are known from the literature. They may be manufactured in particular according to the processes described, for example, in U.S. Pat. No. 6,225,198 or U.S. Pat. No. 5,990,479, in the publications cited therein, and also in the following publications: Dabboussi B. O. et al. “(CdSe)ZnS core-shell quantum dots: synthesis and characterization of a size series of highly luminescent nanocrystallites” Journal of Physical Chemistry B, vol. 101, 1997, pp. 9463-9475 and Peng, Xiaogang et al. “Epitaxial growth of highly luminescent CdSe/CdS core/shell nanocrystals with photostability and electronic accessibility”, Journal of the American Chemical Society, vol. 119, No. 30, pp. 7019-7029.

Pigments with special effects also comprise fluorescent pigments, whether these are substances that are fluorescent in daylight or that produce an ultraviolet fluorescence, phosphorescent pigments, photochromic pigments and thermochromic pigments.

The pigment may be a mineral pigment. The term “mineral pigment” means any pigment that satisfies the definition in Ullmann's encyclopaedia in the chapter on inorganic pigments. Among the mineral pigments that are useful in the present invention, mention may be made of zirconium oxide or cerium oxide, and also iron oxide or chromium oxide, manganese violet, ultramarine blue, chromium hydrate, ferric blue and titanium dioxide. The following mineral pigments may also be used: Ta₂O₅, Ti₃O₅, Ti₂O₃, TiO, ZrO₂ as a mixture with TiO₂, ZrO₂, Nb₂O₅, CeO₂, ZnS.

The pigment may also be a nacreous pigment such as white nacreous pigments, for example mica coated with titanium or with bismuth oxychloride, coloured nacreous pigments such as mica coated with titanium and with iron oxides, mica coated with titanium and especially with ferric blue or chromium oxide, mica coated with titanium and with an organic pigment as defined above, and also nacreous pigments based on bismuth oxychloride. Examples that may be mentioned include the Cellini pigments sold by Engelhard (Mica-TiO₂-lake), Prestige sold by Eckart (Mica-TiO₂) or Colorona sold by Merck (Mica-TiO₂—Fe₂O₃).

In addition to nacres on a mica support, multilayer pigments based on synthetic substrates such as alumina, silica, calcium sodium borosilicate or calcium aluminium borosilicates, and aluminium, may be envisaged.

The size of the pigment that is useful in the context of the present invention is generally between 10 nm and 200 μm, preferably between 20 nm and 80 μm and more preferentially between 30 nm and 50 μm.

According to one particular embodiment, a subject of the invention is a cosmetic composition for coating keratin materials, comprising
at least one compound A and at least one compound B, at least one of the compounds A and B being a silicone compound, the said compounds A and B being capable of reacting together via a hydrosilylation or condensation reaction, or a crosslinking reaction in the presence of a peroxide, when they are placed in contact with each other, and
at least one pigment other than carbon black and iron oxides.

The pigments may be dispersed in the product by means of a dispersant.

The dispersant serves to protect the dispersed particles against agglomeration or flocculation. This dispersant may be a surfactant, an oligomer, a polymer or a mixture of several thereof, bearing one or more functionalities with strong affinity for the surface of the particles to be dispersed. In particular, they can physically or chemically attach to the surface of the pigments. These dispersants also contain at least one functional group that is compatible with or soluble in the continuous medium. In particular, 12-hydroxystearic acid esters and C₈ to C₂₀ fatty acid esters of polyols such as glycerol or diglycerol are used, such as poly(12-hydroxystearic acid) stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As other dispersants that may be used in the composition of the invention, mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The polydihydroxystearic acid and the 12-hydroxystearic acid esters are preferably intended for a hydrocarbon-based or fluorinated medium, whereas the mixtures of oxyethylene/oxypropylene dimethylsiloxane are preferably intended for a silicone medium.

The compositions according to the invention may comprise at least one filler, especially in a content ranging from 0.01% to 50% by weight and preferably ranging from 0.01% to 30% by weight relative to the total weight of each first and second composition or relative to the total weight of the composition when A and B are present in the same composition. The fillers may be mineral or organic and of any form, platelet-shaped, spherical or oblong, irrespective of the crystallographic form (for example lamellar, cubic, hexagonal, orthorhombic, etc.). Mention may be made of talc, mica, silica, silica surface-treated with a hydrophobic agent, kaolin, polyamide powder, for instance Nylon® (Orgasol® from Atochem), poly-β-alanine powder and polyethylene powder, tetrafluoroethylene polymer powders, (Teflon®), lauroyllysine, starch, boron nitride, expanded hollow polymer microspheres such as those made of polyvinylidene chloride/acrylonitrile, for instance Expancel® (Nobel Industrie), acrylic acid copolymers (Polytrap® from Dow Corning) and silicone resin microbeads (for example Tospearls® from Toshiba), elastomeric polyorgano-siloxane particles, precipitated calcium carbonate, magnesium carbonate optionally treated with stearic acid or stearate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

The compositions according to the invention may also contain ingredients commonly used in cosmetics, such as vitamins, thickeners, gelling agents, trace elements, softeners, sequestering agents, fragrances, acidifying or basifying agents, preserving agents, sunscreens, surfactants, antioxidants, fibres and care agents, or mixtures thereof.

The gelling agents that may be used in the compositions according to the invention may be organic or mineral, and polymeric or molecular, hydrophilic or lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name “Bentone 38V®” by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduction in the number of silanol groups present at the surface of the silica. It is especially possible to substitute silanol groups with hydrophobic groups: a hydrophobic silica is then obtained. The hydrophobic groups may be:
trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references Aerosil R812®, R8200 by the company Degussa, Wacker HDX H2000 by Wacker and Cab-O-Sil TS-530® by the company Cabot;
dimethylsilyloxyl or polydimethylsiloxane groups, which are obtained especially by treating fumed silica in the presence of polydimethylsiloxane or dimethyldichlorosilane. Silicas thus treated are known as “silica dimethyl silylate” according to the CTFA (6th edition, 1995). They are sold, for example, under the references Aerosil R972® and Aerosil R974® by the company Degussa, and Cab-O-Sil TS-610® and Cab-O—Sil TS-720® by the company Cabot.

The hydrophobic fumed silica particularly has a particle size that may be nanometric to micrometric, for example ranging from about 5 to 200 nm.

It is also possible to use non-polymeric, molecular organic gelling agents, also known as organogelling agents, associated with a liquid fatty phase (which may be the liquid fatty phase of the composition according to the invention), which are compounds whose molecules are capable of establishing between themselves physical interactions leading to self-aggregation of the molecules with formation of a supramolecular 3D network that is responsible for the gelation of the liquid fatty phase.

The supramolecular network may result from the formation of a network of fibrils (caused by the stacking or aggregation of organogelling molecules), which immobilizes the molecules of the liquid fatty phase.

The ability to form this network of fibrils, and thus to gel, depends on the nature (or chemical class) of the organogelling agent, on the nature of the substituents borne by its molecules for a given chemical class, and on the nature of the liquid fatty phase.

The physical interactions are of diverse nature but exclude co-crystallization. These physical interactions are in particular interactions of self-complementary hydrogen interaction type, π interactions between unsaturated rings, dipolar interactions, coordination bonds with organometallic derivatives, and combinations thereof. In general, each molecule of an organogelling agent can establish several types of physical interaction with a neighbouring molecule. Thus, advantageously, the molecules of the organogelling agents according to the invention comprise at least one group capable of establishing hydrogen bonds and better still at least two groups, at least one aromatic ring and better still at least two aromatic rings, at least one or more ethylenically unsaturated bonds and/or at least one or more asymmetric carbons. Preferably, the groups capable of forming hydrogen bonds are chosen from hydroxyl, carbonyl, amine, carboxylic acid, amide, urea and benzyl groups, and combinations thereof.

The organogelling agent(s) according to the invention is (are) soluble in the liquid fatty phase after heating to obtain a transparent uniform liquid phase. They may be solid or liquid at room temperature and atmospheric pressure.

The molecular organogelling agent(s) that may be used in the composition according to the invention is (are) especially those described in the document “Specialist Surfactants” edited by D. Robb, 1997, pp. 209-263, Chapter 8 by P. Terech, European patent applications EP-A-1 068 854 and EP-A-1 086 945, or alternatively in patent application WO-A-02/47031.

Mention may be made especially, among these organogelling agents, of amides of carboxylic acids, in particular of tricarboxylic acids, for instance cyclohexanetricarboxamides (see European patent application EP-A-1 068 854), diamides with hydrocarbon-based chains each containing from 1 to 22 carbon atoms, for example from 6 to 18 carbon atoms, the said chains being unsubstituted or substituted with at least one substituent chosen from ester, urea and fluoro groups (see patent application EP-A-1 086 945) and especially diamides resulting from the reaction of diamino-cyclohexane, in particular diaminocyclohexane in trans form, and of an acid chloride, for instance N,N′-bis-(dodecanoyl)-1,2-diaminocyclohexane, N-acylamino acid amides, for instance the diamides resulting from the action of an N-acylamino acid with amines containing from 1 to 22 carbon atoms, for instance those described in document WO-93/23008 and especially N-acylglutamic acid amides in which the acyl group represents a C₈ to C₂₂ alkyl chain, such as N-lauroyl-L-glutamic acid dibutylamide, manufactured or sold by the company Ajinomoto under the name GP-1, and mixtures thereof.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC Gel®, SR DMF 10 Gel® and SR DC 556 Gel® from Grant Industries and SF 1204® and JK 113® from General Electric; ethylcellulose, for instance the product sold under the name Ethocel by Dow Chemical; polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, and in particular ethylenediamine/stearyl dilinoleate copolymers such as the product sold under the name Uniclear 100 VG® by the company Arizona Chemical; silicone polyamides of the polyorganosiloxane type, for instance those described in documents U.S. Pat. No. 5,874,069, U.S. Pat. No. 5,919,441, U.S. Pat. No. 6,051,216 and U.S. Pat. No. 5,981,680, for instance those sold under the reference Dow Corning 2-8179 Gellant by the company Dow Corning; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof; block copolymers of “diblock” or “triblock” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type.

It is also possible to use silicone polyamides of the polyorganosiloxane type, such as those described in documents U.S. Pat. No. 5,874,069, U.S. Pat. No. 5,919,441, U.S. Pat. No. 6,051,216 and U.S. Pat. No. 5,981,680.

These silicone polymers may belong to the following two families:
polyorganosiloxanes comprising at least two groups capable of establishing hydrogen interactions, these two groups being located in the polymer chain, and/or
polyorganosiloxanes comprising at least two groups capable of establishing hydrogen interactions, these two groups being located on grafts or branches.

Among the gelling agents that may be used in the compositions according to the invention, mention may also be made of fatty acid esters of dextrin, such as dextrin palmitates, especially the products sold under the name Rheopearl TL® or Rheopearl KL® by the company Chiba Flour.

The lipophilic gelling agents may be present in the compositions according to the invention in a content ranging from 0.05% to 40% by weight, preferably from 0.5% to 20% and better still from 1% to 15% by weight relative to the total weight of each first and second composition.

Hydrophilic or water-soluble gelling agents that may be mentioned include:
homopolymers or copolymers of acrylic or methacrylic acid or the salts and esters thereof, and in particular the products sold under the names Versicol F or Versicol″ by the company Allied Colloid, Ultrahold 8 by the company Ciba-Geigy, and the polyacrylic acids of Synthalen K type;
copolymers of acrylic acid and of acrylamide sold in the form of the sodium salt thereof under the names Reten by the company Hercules, sodium polymethacrylate sold under the name Darvan No. 7 by the company Vanderbilt, and the sodium salts of polyhydroxycarboxylic acids sold under the name Hydagen F by the company Henkel;
polyacrylic acid/alkyl acrylate copolymers of the Pemulen type;
AMPS (polyacrylamidomethylpropanesulfonic acid partially neutralized with ammonia and highly crosslinked) sold by the company Clariant;
AMPS/acrylamide copolymers of the Sepigel or Simulgel type, sold by the company SEPPIC, and

AMPS/polyoxyethylenated alkyl methacrylate copolymers (crosslinked or non-crosslinked); and mixtures thereof.

As other examples of water-soluble gelling polymers, mention may be made of:
proteins, for instance proteins of plant origin, such as wheat or soybean proteins; proteins of animal origin such as keratins, for example keratin hydrolysates and sulfonic keratins;
anionic, cationic, amphoteric or nonionic chitin or chitosan polymers;
non-liposoluble cellulose polymers such as hydroxyethylcellulose, hydroxypropylcellulose, methyl-cellulose, ethylhydroxyethylcellulose and carboxy-methylcellulose, and also quaternized cellulose derivatives;
vinyl polymers, for instance polyvinyl-pyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
associative polyurethanes such as the C₁₆—OE₁₂₀-C₁₆ polymer from the company Servo Delden (sold under the name Ser Ad FX1100, which is a molecule containing urethane functions and having a weight-average molecular weight of 1300), OE being an oxyethylene unit, Rheolate 205 containing urea functions, sold by the company Rheox, or Rheolate 208 or 204 (these polymers being sold in pure form) or DW 1206B from Röhm & Haas, containing a C₂₀ alkyl chain and a urethane bond, sold at a solids content of 20% in water. It is also possible to use solutions or dispersions of these associative polyurethanes, especially in water or in aqueous-alcoholic medium. Examples of such polymers that may be mentioned include Ser Ad FX1010, Ser Ad FX1035 and Ser Ad 1070 from the company Servo Delden, and Rheolate 255, Rheolate 278 and Rheolate 244 sold by the company Rheox. It is also possible to use the product DW 1206F and DW 1206J, and also Acrysol RM 184 or Acrysol 44 from the company Rohm & Haas, or Borchigel LW 44 from the company Borchers;
optionally modified polymers of natural origin, such as:
gum arabics, guar gum, xanthan derivatives and karaya gum;
alginates and carrageenans;
glycoaminoglycans, and hyaluronic acid and its derivatives;
shellac resin, sandarac gum, dammar resins, elemi gums and copal resins;
deoxyribonucleic acid;
mucopolysaccharides such as hyaluronic acid and chondroitin sulfates, and mixtures thereof.

The hydrophilic gelling agents may be present in the composition according to the invention in a content ranging from 0.05% to 20% by weight, preferably from 0.5% to 10% and better still from 0.8% to 5% by weight relative to the total weight of the first and second compositions.

The compositions according to the invention may contain emulsifying surfactants, which are especially present in a proportion ranging from 0.1% to 30% by weight, better still from 1% to 15% and even better still from 2% to 10% relative to the total weight of the composition. These surfactants may be chosen from anionic, nonionic, amphoteric and zwitterionic surfactants. Reference may be made to the document “Encyclopedia of Chemical Technology, Kirk-Othmer”, Volume 22, pp. 333-432, 3rd Edition, 1979, Wiley, for the definition of the properties and functions (emulsifying) of surfactants, in particular pp. 347-377 of this reference, for the anionic and nonionic surfactants.

The surfactants preferentially used in the first and second compositions according to the invention are chosen from:

a) nonionic surfactants with an HLB of greater than or equal to 8 at 25° C., used alone or as a mixture; mention may be made especially of:
oxyethylenated and/or oxypropylenated ethers (which may comprise from 1 to 150 oxyethylene and/or oxypropylene groups) of glycerol;
oxyethylenated and/or oxypropylenated ethers (which may comprise from 1 to 150 oxyethylene and/or oxypropylene groups) of fatty alcohols (especially of C₈-C₂₄ and preferably C₁₂-C₁₈ alcohol), such as oxyethylenated cetearyl alcohol ether containing 30 oxyethylene groups (CTFA name Ceteareth-30) and the oxyethylenated ether of the mixture of C₁₂-C₁₅ fatty alcohols comprising 7 oxyethylene groups (CTFA name C12-15 Pareth-7 sold under the name Neodol 25-7® by Shell Chemicals);
fatty acid esters (especially of a C₈-C₂₄ and preferably C₁₆-C₂₂ acid) of polyethylene glycol (which may comprise from 1 to 150 ethylene glycol units), such as PEG-50 stearate and PEG-40 monostearate sold under the name Myrj 52P by the company ICI Uniqema;
fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name Simulsol 220 ™ by the company SEPPIC; glyceryl stearate polyethoxylated with 30 ethylene oxide groups, for instance the product Tagat S sold by the company Goldschmidt, glyceryl oleate polyethoxylated with 30 ethylene oxide groups, for instance the product Tagat O sold by the company Goldschmidt, glyceryl cocoate polyethoxylated with 30 ethylene oxide groups, for instance the product Varionic LI 13 sold by the company Sherex, glyceryl isostearate polyethoxylated with 30 ethylene oxide groups, for instance the product Tagat L sold by the company Goldschmidt, and glyceryl laurate polyethoxylated with 30 ethylene oxide groups, for instance the product Tagat I from the company Goldschmidt;
fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene groups), for instance polysorbate 60 sold under the name Tween 60 by the company Uniqema;
dimethicone copolyol, such as the product sold under the name Q2-5220 by the company Dow Corning;
dimethicone copolyol benzoate (Finsolv SLB 101 and 201 by the company Finetex);
copolymers of propylene oxide and of ethylene oxide, also known as EO/PO polycondensates, for instance the polyethylene glycol/polypropylene glycol/polyethylene glycol triblock polycondensates sold under the name Synperonic, for instance Synperonic PE/L44 and Synperonic PE/F127, by the company ICI, and mixtures thereof;
and mixtures thereof.
   b) nonionic surfactants with an HLB of less than 8 at 25° C., optionally combined with one or more nonionic surfactants with an HLB of greater than 8 at 25° C., as mentioned above, such as:
saccharide esters and ethers, such as sucrose stearate, sucrose cocoate and sorbitan stearate, and mixtures thereof, for instance Arlatone 2121 sold by the company ICI;
fatty acid esters (especially of a C₈-C₂₄ and preferably C₁₆-C₂₂ acid) of polyols, especially of glycerol or of sorbitol, such as glyceryl stearate, glyceryl stearate such as the product sold under the name Tegin M by the company Goldschmidt, glyceryl laurate such as the product sold under the name Imwitor 312 by the company Hüls, polyglyceryl-2 stearate, sorbitan tristearate or glyceryl ricinoleate;
the mixture of cyclomethicone/dimethicone copolyol sold under the name Q2-3225C by the company Dow Corning.
   c) anionic surfactants such as:
C₁₆-C₃₀ fatty acid salts, especially those derived from amines, for instance triethanolamine stearate;
polyoxyethylenated fatty acid salts, especially those derived from amines or alkali metal salts, and mixtures thereof;
phosphoric esters and salts thereof, such as DEA oleth-10 phosphate (Crodafos N 10N from the company Croda) and cetyl phosphate (Amphisol K from the company DSM Nutritional Products);
sulfosuccinates such as Disodium PEG-5 citrate lauryl sulfosuccinate and Disodium ricinoleamido MEA sulfosuccinate;
alkyl ether sulfates, such as sodium lauryl ether sulfate;
isethionates;
acylglutamates such as Disodium hydrogenated tallow glutamate (Amisoft HS-21 R sold by the company Ajinomoto), and mixtures thereof.

Triethanolamine stearate is most particularly suitable for the invention. This is generally obtained by simple mixing of stearic acid and triethanolamine.

Surfactants that allow an oil-in-water or wax-in-water emulsion to be obtained are preferably used.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, preferably from 5 to 500 and better still from 5 to 150.

They may especially be fibres used in the manufacture of textiles, and especially silk fibre, cotton fibre, wool fibre, flax fibre, cellulose fibre extracted in particular from wood, from plants or from algae, rayon fibre, polyamide (Nylon®) fibre, viscose fibre, acetate fibre, especially rayon acetate fibre, poly(p-phenyleneterephthalamide) (or aramid) fibre, especially Kevlar® fibre, acrylic polymer fibre, especially polymethyl methacrylate fibre or poly(2-hydroxyethyl methacrylate) fibre, polyolefin fibre and especially polyethylene or polypropylene fibre, glass fibre, silica fibre, carbon fibre, especially of carbon in graphite form, polytetrafluoroethylene (such as Teflon®) fibre, insoluble collagen fibre, polyester fibre, polyvinyl chloride fibre or polyvinylidene chloride fibre, polyvinyl alcohol fibre, polyacrylo-nitrile fibre, chitosan fibre, polyurethane fibre, polyethylene phthalate fibre, and fibres formed from a mixture of polymers such as those mentioned above, for instance polyamide/polyester fibres.

The compositions according to the invention may comprise any cosmetic active agent, such as active agents chosen from antioxidants, preserving agents, fragrances, bactericidal or antiperspirant active agents, neutralizers, emollients, moisturizers, vitamins and screening agents, in particular sunscreens.

Needless to say, a person skilled in the art will take care to select this or these optional additional compound(s), and/or the amount thereof, such that the advantageous properties of the corresponding composition according to the invention are not, or are not substantially, adversely affected by the envisaged addition, especially so as not to interfere with the reaction between compounds A and B.

The first and second, and where appropriate third, compositions of the process according to the invention may be, independently, in the form of a suspension, a dispersion, a solution, a gel, an emulsion, especially an oil-in-water (O/W) emulsion, a wax-in-water or water-in-oil (W/O) emulsion or a multiple emulsion (W/O/W or polyol/O/W or O/W/O) or in the form of a cream, a paste, a mousse, a vesicular dispersion, especially of ionic or nonionic lipids, a two-phase or multiphase lotion, a powder or a paste, especially a soft paste.

The process according to the invention may be advantageously used for making up the skin, the lips, the eyelashes and/or the nails depending on the nature of the ingredients used. In particular, the compositions in the process according to the invention may be, independently, in the form of a solid foundation, a lipstick wand or paste, a concealer product, an eye contour product, an eyeliner, a mascara, an eyeshadow, a body makeup product or a skin colouring product.

According to one embodiment, the first and second, and where appropriate third, compositions are lipstick compositions.

According to another embodiment, the first and second, and where appropriate third, compositions are compositions for coating the eyelashes or the eyebrows and more particularly mascaras.

According to another embodiment, the first and second, and where appropriate third, compositions are compositions for coating bodily or facial skin, more particularly compositions for making up bodily or facial skin, for instance foundations or body makeup compositions.

A person skilled in the art may select the appropriate galenical form, and also the method for preparing it, on the basis of his general knowledge, taking into account firstly the nature of the constituents used, especially their solubility in the support, and secondly the intended use of each composition.

The invention is illustrated in greater detail by the examples described below. Unless otherwise mentioned, the amounts indicated are expressed as mass percentages.

### EXAMPLE 1

### Lipsticks

The following mixtures X and Y from Dow Corning are used in compositions 1 and 2:

Part X:

| | | Amount | |
|---|---|---|---|
| Ingredient (INCI name) | CAS N^{o} | (%) | Role |
| Dimethyl Siloxane, | 68083- | 55-95 | Polymer |
| Dimethylvinylsiloxy- | 19-2 | | |
| terminated | | | |
| Silica Silylate | 68909- | 10-40 | Filler |
| | 20-6 | | |
| 1,3-Diethenyl-1,1,3,3- | 68478- | Trace | Catalyst |
| Tetramethyldisiloxane complex | 92-2 | | |
| Tetramethyldivinyldisiloxane | 2627-95-4 | 0.1-1 | Polymer |

Part Y:

| | | Amount | |
|---|---|---|---|
| Ingredient (INCI name) | CAS N^{o} | (%) | Role |
| Dimethyl Siloxane, | 68083- | 55-95 | Polymer |
| Dimethylvinylsiloxy- | 19-2 | | |
| terminated | | | |
| Silica Silylate | 68909- | 10-40 | Filler |
| | 20-6 | | |
| Dimethyl, Methylhydrogen | 68037- | 1-10 | Polymer |
| Siloxane, trimethylsiloxy- | 59-2 | | |
| terminated | | | |

The following compositions are prepared:

### Composition 1

| | Weight % |
|---|---|
| Phenyl trimethicone (DC 556 from Dow | 43.73 |
| Corning) | |
| Part X | 50 |
| Pigments | 6.27 |

### Composition 2

| | Weight % |
|---|---|
| Phenyl trimethicone (DC 556 from Dow | 50 |
| Corning) | |
| Part Y | 50 |

The first and second compositions above are mixed together extemporaneously in a 50/50 proportion, and this mixture is then applied to the lips. After drying for a few minutes, a glossy film that does not transfer is observed on the lips.

### a/ The Gloss of The Film is Measured According to the Following Protocol:

A coat 150 μm thick of the composition is spread onto a Byk Gardner brand contrast card of reference Prüfkarten, Art. 2853, premounted onto a 1 mm glass plate, using an automatic spreader (Bar coater, Sheen). The coat covers at least the black background of the card. When the composition is solid, it is melted, if necessary, on the card after having been spread, so that it covers the black background. Once the composition has been spread, the gloss at 60° is measured on the black background using a Byk Gardner brand glossmeter of reference microTri-Gloss. Thus, four contrast cards are prepared to measure the mean gloss of the composition, and the mean of the four measurements is determined. In order for the measurement to be correct, the standard deviation must be less than or equal to 3%.

The formed film has a mean gloss at 60° equal to 54.

b/ The Transfer of the Film Obtained with the Mixture of the Two Compositions is Evaluated as Follows:

A support (rectangle of 40 mm×70 mm and 3 mm thick) of polyethylene foam that is adhesive on one of the faces, having a density of 33 kg/m³ (sold under the name RE40X70EP3 from the company Joint Technique Lyonnais Ind) is preheated on a hotplate maintained at a temperature of 40° C. in order for the surface of the support to be maintained at a temperature of 33° C.±1° C.

The mixture of the two compositions is applied over the entire non-adhesive surface of the support, by spreading it using a fine brush to obtain a deposit of about 15 μm of the composition, while leaving the support on the hotplate, and the support is then left to dry for 30 minutes.

After drying, the support is bonded via its adhesive face onto an anvil of diameter 20 mm and equipped with a screw pitch. The support/deposit assembly is then cut up using a punch 18 mm in diameter. The anvil is then screwed onto a press (Statif Manuel Imada SV-2 from the company Someco) equipped with a tensile testing machine (Imada DPS-20 from the company Someco).

White photocopier paper of 80 g/m² is placed on the bed of the press and the support/deposit assembly is then pressed on the paper at a pressure of 2.5 kg for 30 seconds. After removing the support/deposit assembly, some of the deposit is transferred onto the paper. The colour of the deposit transferred onto the paper is then measured using a Minolta CR300 colorimeter, the colour being characterized by the L*, a*, b* colorimetric parameters. The calorimetric parameters L*₀, a*₀ and b*₀ of the colour of the plain paper used is determined.

The difference in colour ΔE1 between the colour of the deposit transferred relative to the colour of the plain paper is then determined by means of the following relationship.

ΔE1=\sqrt{(L*−L₀*)²+(a*−a₀*)²+(b*−b₀*)²}

Separately, a total transfer reference is prepared by applying the composition directly onto a paper identical to the one used previously, at room temperature (25° C.), by spreading the composition using a fine brush and so as to obtain a deposit of about 15 μm of the composition, and the deposit is then left to dry for 30 minutes at room temperature (25° C.). After drying, the calorimetric parameters L*′, a*′ and b*′ of the colour of the deposit placed on the paper, corresponding to the reference colour of total transfer, is measured directly. The calorimetric parameters L*′₀, a*′₀ and b*′₀ of the colour of the plain paper used are determined.

The difference in colour ΔE2 between the reference colour of total transfer relative to the colour of the plain paper is then determined by means of the following relationship.

ΔE2=\sqrt{(L*′−L₀*′)²+(a*′−a₀*′)²+(b*−b₀*′)²}

The transfer of the composition, expressed as a percentage, is equal to the ratio:

100×ΔE1/ΔE2

The measurement is performed on 4 supports in succession and the transfer value corresponds to the mean of the 4 measurements obtained with the 4 supports.

The film obtained from the mixture of compositions 1 and 2 has a transfer value of 0%.

### EXAMPLE 2

### Lipsticks

The following mixtures X′ and Y′ from Dow Corning are used in compositions 1 and 2:

Part X′:

| | | Amount | |
|---|---|---|---|
| Ingredient (INCI name) | CAS N^{o} | (%) | Role |
| Bis-Trimethoxysiloxyethyl | PMN87176 | 25-45 | Polymer |
| Tetramethyldisiloxyethyl | | | |
| Dimethicone | | | |
| Silica Silylate | 68909- | 5-20 | filler |
| | 20-6 | | |
| Disiloxane | 107-46-0 | 30-70 | Solvent |

Part Y′:

| Ingredient (INCI | | | |
|---|---|---|---|
| name) | CAS N^{o} | Amounts (%) | Role |
| Disiloxane | 107- | 80-99 | Solvent |
| | 46-0 | | |
| Tetra T Butyl | — | 1-20 | Catalyst |
| Titanate | | | |

In this case, the compounds A and B combined in part Y′ are the same.

The following compositions are prepared

### Composition 1

| | in g |
|---|---|
| Phenyl trimethicone (DC 556 from Dow | 4.88 |
| Corning) | |
| Part X′ | 83.18 |
| Pigments | 2.85 |
| Total | 90.91 |

### Composition 2

| | in g |
|---|---|
| Part Y′ | 9.09 |

The first and second compositions above are mixed together extemporaneously so as to obtain 100 g of mixture, which is then applied to the lips. After drying for a few minutes, a matt film that does not transfer is observed on the lips.

The transfer value of the film of is measured according to the protocol indicated in Example 1: the film has a transfer of 0%.

1. Cosmetic process for coating keratin materials, which consists in applying to the said keratin materials at least one coat of a mixture of a first composition and of a second composition, the first and/or second composition comprising at least one compound A and/or at least one compound B and optionally at least a catalyst or a peroxide; at least one of the compounds A and B being a silicone compound, the said compounds A and B being capable of reacting together via a hydrosilylation reaction or a condensation reaction, or a crosslinking reaction in the presence of a peroxide, when they are placed in contact with each other, provided that the compounds A and B, and the catalyst when present or the peroxide, are not present together in the same compositions, the said mixture being obtained either extemporaneously before application to the keratin materials, or simultaneously with its application to the keratin materials.

2. Cosmetic process for coating keratin materials, the process comprising the application to the said keratin materials:
a. of at least one coat of a first composition;
b. of at least one coat of a second composition; the first and/or second composition comprising at least one compound A and/or at least one compound B and optionally at least a catalyst or a peroxide, at least one of the compounds A and B being a silicone compound, provided that the compounds A and B, and the catalyst when present, or the peroxide, are not present together in the same compositions, the said compounds A and B being capable of reacting together via a hydrosilylation reaction or a condensation reaction, or a crosslinking reaction in the presence of a peroxide, when they are placed in contact with each other.

3. Process according to either of the preceding claims, characterized in that compounds A and B are capable of reacting via hydrosilylation.

4. Process according to claim 3, characterized in that compound A is a polyorganosiloxane comprising a main chain whose unsaturated aliphatic groups are pendent to the main chain (side group) or located at the ends of the main chain of the compound (end group).

5. Process according to one of claims 3, characterized in that compound A is chosen from polyorganosiloxanes comprising at least two unsaturated aliphatic groups linked to a silicon atom.

6. Process according to one of claims 3 to 5, characterized in that compound A is chosen from polyorganosiloxanes comprising siloxane units of formula: 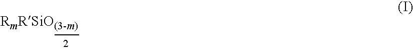
in which:
R represents a linear or cyclic monovalent hydrocarbon-based group containing from 1 to 30 carbon atoms,
m is equal to 1 or 2, and
R′represents a vinyle group or a group R″—CH═CHR′″ in which R″ is a divalent aliphatic hydrocarbon chain containing from 1 to 8 carbon atoms, linked to the silicon atom and R′″ is a hydrogen atom or an alkyl radical, preferably a hydrogen atom.

7. Process according to claim 6, characterized in that R represents an alkyl radical containing from 1 to 10 carbon atoms or a phenyl group and R′ is a vinyl group.

8. Process according to one of claims 3 to 7, characterized in that the polyorganosiloxanes also comprise units of formula 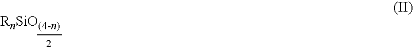
in which R is an alkyl radical containing from 1 to 30 carbon atoms or a phenyl group, and n is equal to 1, 2 or 3.

9. Process according to claim 3, characterized in that compound A is chosen from organic oligomers or polymers and hybrid organic/silicone oligomers or polymers, the said oligomers or polymers bearing at least two reactive unsaturated aliphatic groups, and mixtures thereof.

10. Process according to one of claim 3 to 9, characterized in that compound B is chosen from polyorganosiloxanes comprising at least two free Si—H groups.

11. Process according to one of claims 3 to 10, characterized in that compound B is chosen from organosiloxanes comprising at least one alkylhydrogenosiloxane unit of the following formula: 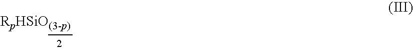
in which:
R represents a linear or cyclic monovalent hydrocarbon-based group containing from 1 to 30 carbon atoms, preferably a methyl group or a phenyl group, and p is equal to 1 or 2.

12. Process according to one of claims 10 to 11, characterized in that compound B comprises at least two alkylhydrogenosiloxane units of formula (H₃C)HSiO and optionally comprises units (H₃C)₂SiO.

13. Process according to one of claims 3 to 12, characterized in that one of the composition contains a platinum-based catalyst.

14. Process according to one of claims 3 to 9 and 11 to 13, characterized in that compound A is a polydimethylsiloxane with vinyl end groups and compound B is a methylhydrogenosiloxane.

15. Process according to one of the preceding claims, characterized in that compound A represents from 0.1% to 95%, preferably from 1% to 90% and better still from 5% to 80% by weight relative to the total weight of the composition comprising it and compound B represents from 0.1% to 95%, preferably from 1% to 90% and better still from 5% to 80% by weight relative to the total weight of the composition comprising it.

16. Process according to one of the preceding claims, characterized in that at least one of the first and second compositions comprises a liquid fatty phase comprising at least one organic solvent or oil chosen from volatile oils and non-volatile oils, and mixtures thereof.

17. Process according to the preceding claim, characterized in that the oil(s) is (are) present in a content ranging from 1% to 90% by weight and preferably from 5% to 50% by weight relative to the total weight of each composition.

18. Process according to claim 16 or 17, characterized in that the non-volatile oil is chosen from:
hydrocarbon-based oils of plant origin, such as triesters of fatty acids and of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, marrow oil, blackcurrant oil, evening primrose oil, millet oil, barley oil, quinoa oil, rye oil, safflower oil, candlenut oil, passionflower oil or musk rose oil; or caprylic/capric acid triglycerides,
synthetic ethers containing from 10 to 40 carbon atoms,
apolar hydrocarbon-based oils, for instance squalene, linear or branched hydrocarbons such as liquid paraffin, liquid petroleum jelly and naphthalene oil, hydrogenated or partially hydrogenated polyisobutene, isoeicosane, squalane, decene/butene copolymers and polybutene/polyisobutene copolymers,
synthetic esters, for instance oils of formula R₁COOR₂ in which R₁, represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, alcohol or polyalcohol octanoates, decanoates or ricinoleates, for instance propylene glycol dioctanoate; hydroxylated esters, for instance isostearyl lactate or diisostearyl malate; and pentaerythritol esters;
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpenta-decanol;
higher fatty acids such as oleic acid, linoleic acid or linolenic acid;
carbonates;
acetates;
citrates;
non-volatile polydimethylsiloxanes (PDMS),
polydimethylsiloxanes comprising alkyl or alkoxy groups, which are pendent and/or at the end of a silicone chain, these groups each containing from 3 to 40 carbon atoms,
phenyl silicones,
optionally fluorinated polyalkylmethylsiloxanes, for instance polymethyltrifluoropropyldimethylsiloxanes,
polyalkylmethylsiloxanes substituted with functional groups such as hydroxyl, thiol and/or amine groups;
polysiloxanes modified with fatty acids, fatty alcohols or polyoxyalkylenes,
and mixtures thereof.

19. Process according to claim 16, 17 or 18, characterized in that the non-volatile oil is chosen from:
esters corresponding to formula (IV) below:
R₁—CO—O—R₂ (IV)
where R₁ represents a linear or branched alkyl radical of 1 to 40 carbon atoms and preferably of 7 to 19 carbon atoms, optionally comprising one or more ethylenic double bonds, and optionally substituted,
R₂ represents a linear or branched alkyl radical of 1 to 40 carbon atoms, preferably of 3 to 30 carbon atoms and better still of 3 to 20 carbon atoms, optionally comprising one or more ethylenic double bonds, and optionally substituted,
phenyl silicones, and
mixtures thereof.

20. Process according to one of claims 16 to 19, characterized in that the non-volatile oil is chosen from:
purcellin oil (cetostearyl octanoate), isopropyl myristate, isopropyl palmitate, C₁₂-C₁₅ alkyl benzoates, hexyl laurate, diisopropyl adipate, isononyl isononanoate, 2-ethylhexyl palmitate or isostearyl isostearate,
phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates,
and mixtures thereof.

21. Process according to one of claims 16 to 20, characterized in that the non-volatile oil is present in a content ranging from 0.1% to 80% by weight, preferably from 1% to 60% by weight, better still from 5% to 50% by weight and even better still from 14% to 40% by weight relative to the total weight of each composition.

22. Process according to any one of the preceding claims, characterized in that at least one of the first and second compositions comprises at least one dyestuff.

23. Process according to one of the preceding claims, characterized in that the first and second, and where appropriate third, compositions are lipstick compositions.

24. Process according to one of claims 1 to 23, characterized in that the first and second, and where appropriate third, compositions are mascara compositions.

25. Cosmetic composition for coating keratin materials, comprising
at least one compound A and at least one compound B, at least one of the compounds A and B being a silicone compound, the said compounds A and B being capable of reacting together via a hydrosilylation reaction or a condensation reaction, or a crosslinking reaction in the presence of a peroxide, when they are placed in contact with each other, and
at least one pigment other than carbon black and iron oxides.

26. Kit for coating keratin materials, comprising at least one compound A and/or at least one compound B and optionally at least a catalyst or a peroxide, at least one of the compounds A and B being a silicone compound, provided that the compounds A and B, and the catalyst when present, or the peroxide, are not present together in the same compositions;
the said compounds A and B being capable of reacting together via a hydrosilylation reaction or a condensation reaction, or a crosslinking reaction in the presence of a peroxide, when they are placed in contact with each other.

27. Kit according to claim 26, characterized in that the first and second compositions are packaged separately in the same packaging article.
